(19) United States
(12) Patent Application Publication
(10) Pub. No.: US 2024/0307390 A1
(43) Pub. Date: Sep. 19, 2024

(54) METHODS AND COMPOSITIONS FOR CANCER TREATMENT BY TARGETING AXL SIGNALING

(71) Applicant: Sanford Burnham Prebys Medical Discovery Institute, La Jolla, CA (US)

(72) Inventors: Charles SPRUCK, San Diego, CA (US); Jia Z. SHEN, La Jolla, CA (US)

(21) Appl. No.: 18/442,658

(22) Filed: Feb. 15, 2024

Related U.S. Application Data
(60) Provisional application No. 63/485,485, filed on Feb. 16, 2023.

(51) Int. Cl. A61K 31/506; A61K 31/4525; A61K 38/46; A61K 49/00; A61P 35/00; C12N 15/113; G01N 33/50

(52) U.S. Cl. CPC A61K 31/506; A61K 31/4525; A61K 38/465; A61K 49/0008; A61P 35/00; C12N 15/113; G01N 33/5011; C12N 2310/14; C12N 2310/141; C12N 2310/531

(57) ABSTRACT

Methods of treating cancer comprising targeting the FBXO7/EYA2-SCFFBXW7 axis, such as via EYA2 Tyr phosphatase inhibitors. As EYA2 acts downstream of SCFFBXW7, FBXO7/EYA2 inhibitors can be effective in blocking immune escape pathways and immunotherapy resistance in Fbxw7 mutant tumors. Furthermore, FBXO7/EYA2 inhibitors can be effective in mitigating AXL signaling and associated immune evasion, thus enhancing antitumor immune responses.

### CROSS-REFERENCE

This application claims the benefit of U.S. Provisional Application No. 63/485,485, filed on Feb. 16, 2023, which is incorporated herein by reference in its entirety.

### INCORPORATION OF SEQUENCE LISTING

The instant application contains a Sequence Listing which has been submitted electronically in XML format and is hereby incorporated by reference in its entirety. Said XML copy, created on Jan. 24, 2024, is named 42256-606_201_SL.xml and is 2,289 bytes in size.

### BACKGROUND OF THE DISCLOSURE

Cancer is a leading cause of death worldwide, and various treatment have been developed to treat cancer including radiation therapy, chemotherapy, or immunotherapy. Immune checkpoint blockade (ICB) therapy is a type of immunotherapy that targets immune checkpoint proteins expressed on the surface of cancer cells and immune cells. The immune checkpoint proteins modulate the immune response by acting as brakes, allowing cancer cells to evade the immune system and continue to grow (e.g., immune evasion).

### SUMMARY

Provided herein are methods and pharmaceutical compositions for treating cancer in a subject by modulating the expression of FBXO7, EYA2, or both in cancer cells to enhance the effectiveness of the immune-based therapies (e.g., immune checkpoint blockade therapy).

Provided herein is a method of treating cancer in a subject comprising administering a therapeutically effective amount of treatment to the subject, wherein the treatment comprises i) a F-box only protein 7 (FBXO7) inhibitor, an Eyes absent homology 2 (EYA2) inhibitor, or both, ii) a chemical entity that inhibits the interaction between FBXO7 and EYA2, or iii) a chemical entity that interferes with the stabilization of EYA2 by FBXO7. In some embodiments, the treatment comprises the FBXO7 inhibitor. In some embodiments, the treatment comprises the EYA2 inhibitor. In some embodiments, the treatment comprises the FBXO7 inhibitor and the EYA2 inhibitor. In some embodiments, the EYA2 inhibitor is an EYA2 Tyr phosphatase inhibitor. In some embodiments, the EYA2 Tyr phosphatase inhibitor is:

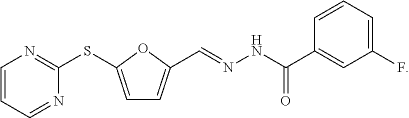 [SMILES: O=C(N/N=C/c1ccc(Sc2ncccn2)o1)c1cccc(F)c1]

In some embodiments, the EYA2 Tyr phosphatase inhibitor is:

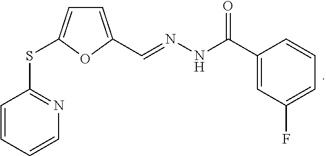 [SMILES: O=C(N/N=C/c1ccc(Sc2ccccn2)o1)c1cccc(F)c1 ; "."]

In some embodiments, the FBXO7 inhibitor is a knockdown mechanism comprising at least one of an RNA interference (RNAi), a small interfering RNA (siRNA), a short hairpin RNA (shRNA), a hairpin siRNA, a precursor microRNA (pre-miRNA) or a microRNA (miRNA), a zinc finger nuclease (ZFN), a bacterial RNA-guided endonuclease, and/or a TAL-effector nuclease (TALEN) directed towards FBXO7 gene. In some embodiments, the knockdown mechanism comprises siRNA. In some embodiments, the knockdown mechanism comprises shRNA. In some embodiments, the EYA2 inhibitor is a knockdown mechanism comprising at least one of an RNA interference (RNAi), a small interfering RNA (siRNA), a short hairpin RNA (shRNA), a hairpin siRNA, a precursor microRNA (pre-miRNA), a microRNA (miRNA), a zinc finger nuclease (ZFN), a bacterial RNA-guided endonuclease, and/or a TAL-effector nuclease (TALEN) directed towards EYA2 gene. In some embodiments, the knockdown mechanism comprises siRNA. In some embodiments, the bacterial RNA-guided endonuclease comprises a Cas protein, and a guide nucleic acid molecule configured to form a complex with the Cas protein. In some embodiments, the Cas protein further comprises a functional domain, wherein the functional domain modulates expression of a target gene. In some embodiments, the target gene comprises FBXO7 or EYA2. In some embodiments, the functional domain comprises a transcriptional activation domain or a transcriptional repression domain. In some embodiments, the transcriptional repression domain comprises a DNMT protein such as DNMT1, DNMT3A, DNMT3B, DNMT3L, a histone deacetylase (HDAC), a histone acetyltransferase (HAT), a histone methylase, or an enzyme that SUMOylates or biotinylates a histone and/or other enzyme domain that allows post-translation histone modification regulated gene repression. In some embodiments, the Cas protein comprises a Cas3, a Cas9, or a Cas10. In some embodiments, the Cas protein is a deactivated Cas. In some embodiments, the method further comprises administering an immune-based therapy to the subject. In some embodiments, the method further comprises administrating a therapeutically effective amount of a programmed cell death protein 1 (PD-1) inhibitor. In some embodiments, the treatment comprises the EYA2 inhibitor and the PD-1 inhibitor. In some embodiments, the PD-1 inhibitor is an anti-PD-1 antibody. In some embodiments, the anti-PD-1 antibody is nivolumab or pembrolizumab. In some embodiments, the cancer is a tumor. In some embodiments, the tumor is a Fbxw7 mutant tumor. In some embodiments, the subject is a mammal or an animal model. In some embodiments, the subject is a mammal, and the mammal is a human. In some embodiments, the chemical entity is a small molecule, an antibody, an RNA interference (RNAi) polynucleotide, a small interfering RNA (siRNA), short hairpin RNA (shRNA), a hairpin siRNA, a precursor microRNA (pre-miRNA), a microRNA (miRNA), a zinc finger nuclease (ZFN), a bacterial RNA-guided endonuclease, a TAL-effector nuclease (TALEN) or an antisense oligonucleotide (ASO) that is complementary to an mRNA sequence.

Provided herein is a method of suppressing FBXO7/EYA2-activation in a cancer cell, comprising (i) contacting the cancer cell with an inhibitor of F-box only protein 7 (FBXO7), or (ii) contacting the cancer cell with an inhibitor of F-box only protein 7 (FBXO7) synthesis. In some embodiments, the inhibitor prevents or decreases AXL signaling in the cancer cell. In some embodiments, the inhibitor of FBXO7 is a small molecule or an antibody. In some embodiments, the inhibitor of FBXO7 synthesis is an RNA interference (RNAi) polynucleotide, a small interfering RNA (siRNA), short hairpin RNA (shRNA), a hairpin siRNA, a precursor microRNA (pre-miRNA), a microRNA (miRNA), a zinc finger nuclease (ZFN), a bacterial RNA-guided endonuclease, a TAL-effector nuclease (TALEN) or an antisense oligonucleotide (ASO) that is complementary to an mRNA sequence. In some embodiments, the inhibitor of FBXO7 synthesis is siRNA. In some embodiments, the inhibitor of FBXO7 synthesis is shRNA. In some embodiments, the inhibitor of FBXO7 synthesis is an ASO. In some embodiments, the inhibitor of FBXO7 synthesis is a siRNA, wherein the siRNA hybridizes with an mRNA. In some embodiments, the inhibitor of FBXO7 synthesis is a CRISPR-Cas9 complex comprising a Cas9 protein or a polynucleotide encoding the Cas9 protein, and a guide RNA or a polynucleotide encoding the guide RNA, wherein the guide RNA hybridizes with a nucleic acid sequence. In some embodiments, the nucleic acid sequence is a DNA sequence. In some embodiments, the inhibitor of FBXO7 synthesis is a Transcription Activator-like Effector Nuclease (TALEN) or a polynucleotide encoding the TALEN. In some embodiments, the method further comprises administrating a therapeutically effective amount of a programmed cell death protein 1 (PD-1) inhibitor. In some embodiments, the treatment comprises the inhibitor of FBXO7 and the PD-1 inhibitor. In some embodiments, the PD-1 inhibitor is an anti-PD-1 antibody. In some embodiments, the anti-PD-1 antibody is nivolumab or pembrolizumab. In some embodiments, the cancer cell is a tumor cell. In some embodiments, the tumor cell is a Fbxw7 mutant tumor cell. In some embodiments, the cancer cell is in a mammal. In some embodiments, the mammal is a human.

Provided herein is a method of treating cancer in a patient, comprising (i) administering a therapeutically effective amount of an inhibitor of F-box only protein 7 (FBXO7), or (ii) administering a therapeutically effective amount of an inhibitor of F-box only protein 7 (FBXO7) synthesis, to the patient. In some embodiments, the inhibitor prevents or decreases AXL signaling in the cancer cell. In some embodiments, the inhibitor of FBXO7 is a small molecule or an antibody. In some embodiments, the inhibitor of FBXO7 synthesis is an RNA interference (RNAi) polynucleotide, a small interfering RNA (siRNA), short hairpin RNA (shRNA), a hairpin siRNA, a precursor microRNA (pre-miRNA), a microRNA (miRNA), a zinc finger nuclease (ZFN), a bacterial RNA-guided endonuclease, a TAL-effector nuclease (TALEN) or an antisense oligonucleotide (ASO) that is complementary to an mRNA sequence. In some embodiments, the inhibitor of FBXO7 synthesis is siRNA. In some embodiments, the inhibitor of FBXO7 synthesis is shRNA. In some embodiments, the inhibitor of FBXO7 synthesis is an ASO. In some embodiments, the inhibitor of FBXO7 synthesis is a siRNA, wherein the siRNA hybridizes with an mRNA. In some embodiments, the inhibitor of FBXO7 synthesis is a CRISPR-Cas9 complex comprising a Cas9 protein or a polynucleotide encoding the Cas9 protein, and a guide RNA or a polynucleotide encoding the guide RNA, wherein the guide RNA hybridizes with a nucleic acid sequence. In some embodiments, the nucleic acid sequence is a DNA sequence. In some embodiments, the inhibitor of FBXO7 synthesis is a Transcription Activator-like Effector Nuclease (TALEN) or a polynucleotide encoding the TALEN. In some embodiments, the method further comprises administrating a therapeutically effective amount of a programmed cell death protein 1 (PD-1) inhibitor. In some embodiments, the treatment comprises the inhibitor of FBXO7 and the PD-1 inhibitor. In some embodiments, the PD-1 inhibitor is an anti-PD-1 antibody. In some embodiments, the anti-PD-1 antibody is nivolumab or pembrolizumab. In some embodiments, the cancer is a tumor. In some embodiments, the tumor is a Fbxw7 mutant tumor. In some embodiments, the subject is a mammal. In some embodiments, the mammal is a human.

Provided herein is a pharmaceutical composition for suppressing FBXO7/EYA2-activation in a cancer cell, comprising (i) a therapeutically effective amount of an inhibitor of F-box only protein 7 (FBXO7), or (ii) a therapeutically effective amount of an inhibitor of F-box only protein 7 (FBXO7) synthesis. In some embodiments, the inhibitor prevents or decreases AXL signaling in the cancer cell. In some embodiments, the inhibitor of FBXO7 is a small molecule or an antibody. In some embodiments, the inhibitor of FBXO7 synthesis is an RNA interference (RNAi) polynucleotide, a small interfering RNA (siRNA), short hairpin RNA (shRNA), a hairpin siRNA, a precursor microRNA (pre-miRNA), a microRNA (miRNA), a zinc finger nuclease (ZFN), a bacterial RNA-guided endonuclease, a TAL-effector nuclease (TALEN) or an antisense oligonucleotide (ASO) that is complementary to an mRNA sequence. In some embodiments, the inhibitor of FBXO7 synthesis is siRNA. In some embodiments, the inhibitor of FBXO7 synthesis is shRNA. In some embodiments, the inhibitor of FBXO7 synthesis is an ASO. In some embodiments, the inhibitor of FBXO7 synthesis is a siRNA, wherein the siRNA hybridizes with an mRNA. In some embodiments, the inhibitor of FBXO7 synthesis is a CRISPR-Cas9 complex comprising a Cas9 protein or a polynucleotide encoding the Cas9 protein, and a guide RNA or a polynucleotide encoding the guide RNA, wherein the guide RNA hybridizes with a nucleic acid sequence. In some embodiments, the nucleic acid sequence is a DNA sequence. In some embodiments, the inhibitor of FBXO7 synthesis is a Transcription Activator-like Effector Nuclease (TALEN) or a polynucleotide encoding the TALEN. In some embodiments, the cancer cell is a tumor cell. In some embodiments, the tumor cell is a Fbxw7 mutant tumor cell. In some embodiments, the cancer cell is in a mammal. In some embodiments, the mammal is a human.

Provided herein is a method for identifying an agent for treating immune checkpoint blockade therapy resistant cancer, comprising: (a) contacting F-box only protein 7 (FBXO7) with a test agent; (b) determining whether the test agent interacts with FBXO7; and (c) subjecting the test agent to an in vitro, ex vivo or in vivo test, wherein a test agent that interacts with FBXO7 reduces or prevents cell proliferation. In some embodiments, determining whether the test agent interacts with FBXO7 comprising measuring a Green Fluorescent Protein (GFP) signal. In some embodiments, the GFP signal shows reduction over an increasing dose of the test agent. In some embodiments, determining whether the test agent interacts with FBXO7 comprises measuring growth of cells over time. In some embodiments, there is a decrease of growth of cells over time relative to nontreatment. In some embodiments, determining whether the test agent interacts with FBXO7 comprises measuring tumor size in one or more mice treated with the test agent. In some embodiments, there is a decrease of tumor size in at least one or more mice treated with the test agent relative to nontreatment. In some embodiments, determining whether the test agent interacts with FBXO7 comprises measuring levels of JUN, MYC, ZEB1, TUBA1C, CDK6, AKT3, or GAS6 protein. In some embodiments, there is an abolition or decrease of protein level relative to nontreatment.

Provided herein is a method for identifying an agent for treating or preventing a tumor growth in a patient having cancer, comprising subjecting a test agent to an in vitro, ex vivo or in vivo model of tumor growth, wherein the test agent interacts with F-box only protein 7 (FBXO7).

Further provided herein is a method for identifying an agent for treating or preventing a tumor growth in a patient having cancer, comprising: (a) contacting a mammalian cell that expresses F-box only protein 7 (FBXO7) with a test agent; (b) determining the activity of FBXO7 in the cell relative to a similar cell that has not been contacted with the test agent; and (c) identifying the agent as an agent that reduces cell proliferation, if the activity of FBXO7 in step (b) is reduced in the cell that has been contacted with the test agent relative the similar cell that has not been contacted with the test agent.

Provided herein is a method of identifying an agent for treating immune checkpoint blockade therapy resistant cancer, comprising: (a) contacting a cell comprising F-box only protein 7 (FBXO7) operably linked to a reporter gene with a test agent; (b) determining the level of expression of the reporter gene; and (c) subjecting the test agent that reduces the level of expression of the reporter gene to an in vitro, ex vivo or in vivo test. In some embodiments, determining whether the test agent interacts with FBXO7 comprises measuring levels of at least one of JUN, MYC, ZEB1, TUBA1C, CDK6, AKT3, and GAS6 protein. In some embodiments, the method further comprises obtaining an output value.

Provided herein is a method of screening a compound library to identify compounds which bind to F-box only protein 7 (FBXO7) for treating or preventing a tumor growth in a patient having cancer, comprising: (a) obtaining a library comprising a plurality of compound structures; (b) performing the method of claim 96 with compounds from the compound library; (c) obtaining a mean output value; and (d) ordering the compounds based on the output value. In some embodiments, the method further comprises selecting compounds that are at least one standard deviation from the mean.

Provided herein is a method of screening a compound library to identify compounds which bind to F-box only protein 7 (FBXO7) for treating or preventing immune checkpoint blockade therapy resistant cancer, comprising: (a) obtaining a library comprising a plurality of compound structures; (b) performing molecular dynamic simulations on FBXO7 crystal structure; (c) generating a FBXO7 structure using RMSD (root-mean-square-difference) clustering of related residues from the molecular dynamic simulations; (d) docking compounds to the FBXO7 structure from step c; and (e) identifying compounds which bind to the FBXO7 structure with a desired affinity. In some embodiments, the docking compounds to the FBXO7 structure comprises docking the compounds to regions of the FBXO7 Eyes Absent Homolog 2 (EYA2) interaction domain.

Provided herein is a method comprising contacting a tumor cell with (i) an inhibitor of an inhibitor of F-box only protein 7 (FBXO7), or (ii) an inhibitor of F-box only protein 7 (FBXO7) synthesis; and measuring AXL expression.

Further provided herein is a method of providing a synergistic combination of pharmaceutical compounds to treat cancer in a subject in need thereof comprising, administering a therapeutically effective amount of an EYA2 inhibitor or a FBXO7 inhibitor and a therapeutically effective amount of an anti-PD1 compound to the subject, wherein the therapeutically effective amount of the EYA2 phosphatase inhibitor or the FBXO7 inhibitor and the therapeutically effective amount of the anti-PD1 compound provide a more than an additive treatment effect on the subject's cancer than the administration of the therapeutically effective amount of the EYA2 phosphatase inhibitor or FBXO7 inhibitor alone and the administration of the therapeutically effective amount of the anti-PD1 compound alone.

Provided herein is a method of reducing immunotherapy resistance comprising, identifying a subject having at least one Fbxw7 mutant tumor and administering a therapeutically effective amount of an EYA2 inhibitor or a therapeutically effective amount an FBXO7 inhibitor to the subject. In some embodiments, the subject is a human.

Provided herein is a method of preventing metastasis progression or targeting metastatic tumors in a subject comprising administering a therapeutically effective amount of treatment to the subject, wherein the treatment comprises i) a F-box only protein 7 (FBXO7) inhibitor, an Eyes absent homology 2 (EYA2) inhibitor, or both, ii) a chemical entity that inhibits the interaction between FBXO7 and EYA2, or iii) a chemical entity that interferes with the stabilization of EYA2 by FBXO7. In some embodiments, the treatment comprises the FBXO7 inhibitor. In some embodiments, the treatment comprises the EYA2 phosphatase inhibitor. In some embodiments, the treatment comprises the FBXO7 inhibitor and the EYA2 inhibitor. In some embodiments, the EYA2 inhibitor is an EYA2 Tyr phosphatase inhibitor. In some embodiments, the EYA2 Tyr phosphatase inhibitor is:

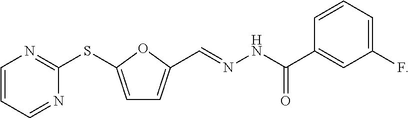 [SMILES: O=C(N/N=C/c1ccc(Sc2ncccn2)o1)c1cccc(F)c1]

In some embodiments, the EYA2 Tyr phosphatase inhibitor is:

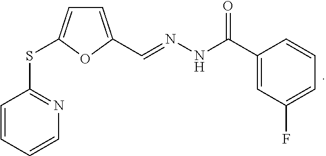 [SMILES: O=C(N/N=C/c1ccc(Sc2ccccn2)o1)c1cccc(F)c1 ; "."]

In some embodiments, the FBXO7 inhibitor is a knockdown mechanism comprising at least one of an RNA interference (RNAi), a small interfering RNA (siRNA), a short hairpin RNA (shRNA), a hairpin siRNA, a precursor microRNA (pre-miRNA) or a microRNA (miRNA), a zinc finger nuclease (ZFN), a bacterial RNA-guided endonuclease, and/or a TAL-effector nuclease (TALEN) directed towards FBXO7 gene. In some embodiments, the knockdown mechanism comprises siRNA. In some embodiments, the knockdown mechanism comprises shRNA. In some embodiments, the EYA2 phosphatase inhibitor is a knockdown mechanism comprising at least one of an RNA interference (RNAi), a small interfering RNA (siRNA), a short hairpin RNA (shRNA), a hairpin siRNA, a precursor microRNA (pre-miRNA), a microRNA (miRNA), a zinc finger nuclease (ZFN), a bacterial RNA-guided endonuclease, and/or a TAL-effector nuclease (TALEN) directed towards EYA2 gene. In some embodiments, the knockdown mechanism comprises siRNA. In some embodiments, the bacterial RNA-guided endonuclease comprises a Cas protein, and a guide nucleic acid molecule configured to form a complex with the Cas protein. In some embodiments, the Cas protein further comprises a functional domain, wherein the functional domain modulates expression of a target gene. In some embodiments, the target gene comprises FBXO7 or EYA2. In some embodiments, the functional domain comprises a transcriptional activation domain or a transcriptional repression domain. In some embodiments, the transcriptional repression domain comprises a DNMT protein such as DNMT1, DNMT3A, DNMT3B, DNMT3L, a histone deacetylase (HDAC), a histone acetyltransferase (HAT), a histone methylase, or an enzyme that SUMOylates or biotinylates a histone and/or other enzyme domain that allows post-translation histone modification regulated gene repression. In some embodiments, the Cas protein comprises a Cas3, a Cas9, or a Cas10. In some embodiments, the Cas protein is a deactivated Cas. In some embodiments, the method further comprises administering an immune-based therapy to the subject. In some embodiments, the method further comprises administrating a therapeutically effective amount of a programmed cell death protein 1 (PD-1) inhibitor. In some embodiments, the treatment comprises the EYA2 phosphatase inhibitor and the PD-1 inhibitor. In some embodiments, the PD-1 inhibitor is an anti-PD-1 antibody. In some embodiments, the anti-PD-1 antibody is nivolumab or pembrolizumab. In some embodiments, the cancer is a tumor. In some embodiments, the tumor is a Fbxw7 mutant tumor. In some embodiments, the subject is a mammal or an animal model. In some embodiments, the subject is a mammal, and the mammal is a human. In some embodiments, the chemical entity is a small molecule, an antibody, an RNA interference (RNAi) polynucleotide, a small interfering RNA (siRNA), short hairpin RNA (shRNA), a hairpin siRNA, a precursor microRNA (pre-miRNA), a microRNA (miRNA), a zinc finger nuclease (ZFN), a bacterial RNA-guided endonuclease, a TAL-effector nuclease (TALEN) or an antisense oligonucleotide (ASO) that is complementary to an mRNA sequence.

Provided herein is a method of screening a compound library to identify compounds which inhibit an interaction between FBXO7 and EYA2, comprising: (a) obtaining a library comprising a plurality of compounds; (b) submitting each compound of the compound library to an in vitro assay reporting a signal, wherein the signal indicates the compound inhibits an interaction between FBXO7 and EYA2; (c) measuring the signal over a range of increasing concentration of the compound; (d) obtaining an EC₅₀ or IC₅₀ output value; and (e) ordering the compounds based on the output value. In some embodiments, the signal indicates a GAS6 protein level. In some embodiments, the signal indicates a EYA2 phosphatase activity level. In some embodiments, the EYA2 phosphatase activity level is determined using fluorescence intensity. In some embodiments, the EYA2 phosphatase activity level is determined by measuring the fluorescence intensity of 3-O-methylfluorescein.

### INCORPORATION BY REFERENCE

All publications, patents, and patent applications mentioned in this specification and exhibits are herein incorporated by reference to the same extent as if each individual publication, patent, or patent application was specifically and individually indicated to be incorporated by reference. Additionally, this application relates to the following publication Shen et al., 2022, Molecular Cell 82, 1123-1139 which is incorporated herein by reference in its entirety.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a schematic of mesenchymal maintenance regulator screen. FIG. 1B shows results of BT-20 cell screen plotted as gene rank for change in E-cadherin vs. KRT14 levels. FBXO7, positive (CDC2), and negative (non-targeting siRNA) controls are highlighted (left panel). FIGS. 1C and 1D show representative immunofluorescence (IF) images of E-cadherin (FIG. 1C) and Vimentin (FIG. 1D) in control and FBXO7 KD MDA-MB-231 cells. FIG. 1E shows immunoblots of indicated proteins in control and FBXO7 KD cancer cells. FIG. 1F shows representative images of control and FBXO7 KD BT-549 cells (left panels). FIG. 1G shows a volcano plot of differentially expressed genes in control and FBXO7 KD MDA-MB-231 cells which shows significantly upregulated (red) and downregulated (blue) genes in FBXO7 KD cells. FIG. 1H shows a heatmap of mesenchymal and immune response genes from RNA-seq for control and FBXO7 KD MDA-MB-231 cells. FIG. 1I represents a RT-qPCR analysis of mesenchymal and immune response genes in control and FBXO7 KD MDA-MB-231 cells. FIG. 1J represents an ELISA quantification of IFNβ and CXCL10 secreted from control and FBXO7 KD MDA-MB-231 cells. FIG. 1K shows a flow cytometry analysis of HLA-A/B/C surface expression on control and FBXO7 KD MDA-MB-231 cells. FIG. 1L represents significantly enriched gene sets in gene set enrichment analysis (GSEA) for FBXO7 KD RNA-seq which shows gene sets increased (red) and decreased (blue) in FBXO7 KD cells. FIG. 1M represents GSEA enrichment plots for select gene sets by RNA-seq (FBXO7 KD vs. control KD).

FIG. 2A shows a mass spectrometry analysis of FBXO7-interacting proteins. FIG. 2B shows a Venn diagram of mass spectrometry results and corresponding FBXO7-interacting proteins. FIG. 2C represents co-IP of endogenous FBXO7 with EYA2. FIG. 2D shows an in-vitro pull-down of recombinant FBXO7 with Tomm20, EYA2, and SIX1. FIG. 2E represents co-immunoprecipitation (IP) of HA-EYA2 and deletion mutants with endogenous FBXO7 in MDA-MB-231 cells. FIG. 2F shows co-IP of endogenous EYA2 with Myc-FBXO7 or Myc-ΔF-FBXO7 in HEK293T cells. FIG. 2G shows representative images and RT-qPCR analysis of indicated genes in control and EYA2 KD MDA-MB-231 cells. FIG. 2H shows a volcano plot of significantly upregulated and downregulated genes in EYA2 KD RNA-seq data. FIG. 2I shows a significantly enriched gene sets in GSEA for EYA2 KD RNA-seq. FIG. 2J shows immunoblots of indicated proteins in dox-inducible Flag-FBXO7 HeLa cells. FIG. 2K shows immunoblots of indicated proteins in cytoplasmic and nuclear fractions of cells in FIG. 2J. FIG. 2L shows representative IF images of Flag-EYA2 in control and Myc-FBXO7-expressing MDA-MB-231 cells. FIGS. 2M and 2N represent EYA2 stability in control and FBXO7-expressing MCF7 cells (FIG. 2M) and control and FBXO7 KD MDA-MB-231 cells (FIG. 2N)

FIG. 3A shows an immunoblot of polyubiquitylated (pUb) Flag-EYA2 in HEK293T cells transfected with the indicated vectors. Input proteins are shown. FIG. 3B shows co-IP of endogenous EYA2 with the indicated F-box proteins. FIG. 3C represents a putative EYA2 CPD motif across species and compared with other SCF^{FBXW7} substrates. FIG. 3D shows an immunoblot of pUb Flag-EYA2 in HEK293T cells transfected with the indicated vectors and treated with GSK3βi. FIG. 3E shows co-IP of WT and CPD mutant Flag-EYA2-2A with GST-FBXW7. FIGS. 3F and 3G represent WT and Flag-EYA2-2A stability in MDA-MB-231 cells (FIG. 3F) and endogenous EYA2 in WT and Fbxw7 KO HCT116 cells (FIG. 3G). FIG. 3H shows an immunoblot of pUb Flag-EYA2 in WT and Fbxw7 KO HCT116 cells. FIG. 3I shows in vitro binding of SCFFBXW7 with un-phosphorylated (A) or phosphorylated (B) EYA2 CPD peptide. FIG. 3J shows in vitro ubiquitylation reactions of SCFFBXW7 with EYA2 with/without FBXO7. FIG. 3K represents flag-EYA2 stability in MDA-MB-231 cells treated with vehicle (DMSO) or GSK3βi. FIG. 3L shows a co-IP of Flag-FBXW7 with endogenous EYA2 in MDA-MB-231 cells transfected with the indicated FBXO7 vectors. FIG. 3M shows an immunoblot of pUb Flag-EYA2 in HEK293T cells transfected with the indicated vectors. Input proteins shown. FIG. 3N shows a RT-qPCR analysis of indicated genes in WT and Fbxw7 KO HCT116 cells. FIG. 3O represents a model of FBXO7 inhibition of GSK3β-SCFFBXW7-mediated degradation of EYA2.

FIG. 4A shows heatmaps of EYA2 ChIP-seq signals in control and FBXO7 KD MDA-MB-231 cells. FIG. 4B shows a ChIP-seq enrichment profiles of EYA2 peaks in control and FBXO7 KD MDA-MB-231 cells. FIG. 4C shows a pathway enrichment map of significantly enriched gene sets in GSEA differentially bound by EYA2 in FBXO7 KD ChIP-seq. FIG. 4D shows EYA2 binding Gas6 promoter in control and FBXO7 KD MDA-MB-231 cells. FIGS. 4E and 4F represent ChIP analysis of Flag-EYA2 (FIG. 4E) and Flag-SIX1 (FIG. 4F) binding Gas6 promoter in MDA-MB-231 cells transfected with the indicated siRNAs. FIGS. 4G-4I show GAS6 expression in MCF7 cells transfected with indicated vectors (FIG. 4G) and MDA-MB-231 cells expressing indicated shRNAs (FIG. 4H) or treated with vehicle or EYA2i (FIG. 4I). FIGS. 4J and 4K show immunoblots of AXL signaling proteins in MDA-MB-231 cells transfected with control or FBXO7 siRNA (FIG. 4J) or treated with vehicle or EYA2i (FIG. 4K). FIG. 4L shows an RT-qPCR analysis of indicated genes in control, FBXO7 KD, and FBXO7 KD+GAS6 MDA-MB-231 cells and immunoblots of indicated proteins. FIG. 4M shows representative IF images of AXL in control and FBXO7 KD MDA-MB-231 cells. FIG. 4N shows a RT-qPCR analysis of indicated genes in control and AXL KD MDA-MB-231 cells. FIG. 4O shows a RT-qPCR analysis of indicated genes in control, FBXO7 KD, and FBXO7 KD+Flag-SOCS1 MDA-MB-231.

FIGS. 5A and 5B show a gap closure assay of control, FBXO7 KD, and FBXO7 KD+GAS6 MDA-MB-231 (FIG. 5A) and BT-549 (FIG. 5B) cells. FIGS. 5C and 5D show the migration (FIG. 5C) and invasion (FIG. 5D) of indicated control, FBXO7 KD, and FBXO7 KD+GAS6 cells. FIG. 5E shows an extreme limiting dilution analysis (ELDA) for indicated control, FBXO7 KD, and FBXO7 KD+GAS6 cells. FIG. 5F shows representative images (left panels) and quantification (right panel) of tumor spheres of indicated control, FBXO7 KD, and FBXO7 KD+GAS6 cells. FIGS. 5G and 5H show viabilities of indicated control, FBXO7 KD, or FBXO7 KD+GAS6 (FIG. 5G) or vehicle or EYA2i treated (FIG. 5H) cells upon doxorubicin treatment.

FIGS. 6A and 6B show growth curves (FIG. 6A) and images (FIG. 6B) of control, FBXO7 KD, and FBXO7 KD+GAS6 MDA-MB-231 breast tumors in immunocompromised mice. FIG. 6C shows a RT-qPCR analysis of indicated genes in tumors in (FIG. 6B). FIG. 6D shows representative images of lung metastases (arrows, inset) in mice in (FIG. 6A). FIGS. 6E and 6F show growth curves (FIG. 6E) and images (FIG. 6F) of control and GAS6-expressing MDA-MB-231-luc breast tumors in immunocompromised mice treated with vehicle or EYA2i. FIG. 6G shows images and quantification of total radiance in lungs of immunocompromised mice 29 days post-intracardiac injection of control or GAS6-expressing MDA-MB-231-luc cells treated with vehicle or EYA2i. FIG. 6H shows growth curves of 4T1-luc breast tumors in syngeneic immunocompetent mice treated with vehicle or EYA2i and IgG isotype (control) or anti-PD-1. FIG. 6I shows representative bioluminescence images of mice in (FIG. 6H) at day 32. FIG. 6J shows a flow cytometry analysis of indicated infiltrating immune cells in 4T1-luc breast tumors in (FIG. 6H) at day 32. FIG. 6K shows representative IHC images and quantification of CD8+ T cells and NK cells in tumors in (FIG. 6H). FIG. 6L shows a flow cytometry analysis of MHC class I (H-2Kd) surface expression on 4T1-luc tumor cells in (FIG. 6H). FIG. 6M shows survival curves for mice in (FIG. 6H).

FIG. 7A shows FBXO7 expression in the indicated cancer types relative to normal adjacent tissue (Oncomine, Compendia Bioscience, Ann Arbor, MI). FIG. 7B shows violin plots of FBXO7 and EYA2 expression in breast cancer subtypes. FIG. 7C shows raincloud plots of FBXO7 and EYA2 expression in basal-like breast cancers and TNBCs. FIG. 7D shows FBXO7 expression linked to patient survival using Tumor Immune Dysfunction and Exclusion (TIDE) framework in breast cancer dataset. FIGS. 7E-7G show a correlation analysis of FBXO7 (FIG. 7E) or FBXW7 expression (FIG. 7F), or Fbxw7 alterations (FIG. 7G), and activities of canonical pathways from the Molecular Signatures Database in TCGA pan-cancer gene expression dataset. FIGS. 7H and 7I show a correlation analysis of FBXO7 (FIG. 7H) or FBXW7 (FIG. 7I) expression and z-scores for TGFβ receptor signaling in EMT in indicated cancer types from TCGA dataset. FIG. 7J shows FBXO7 expression linked to CTL abundance using TIDE framework in breast cancer dataset. FIG. 7K shows FBXO7-immune gene signature z-scores in responder and non-responder groups of patients treated with anti-PD-1 or TIL therapy in the indicated datasets. FIG. 7L shows a model of FBXO7 maintenance of mesenchymal and immune evasion phenotypes of cancer cells.

### DETAILED DESCRIPTION OF THE DISCLOSURE

### Overview

AXL, a member of the TAM (TYRO3/AXL/MERTK) family of receptor tyrosine kinase, signaling is frequently overexpressed in immunotherapy resistance cancer cells (e.g., triple-negative breast cancers). Provided herein, in some aspects, are methods, pharmaceutical compositions, kits and dosing regimens for treating cancer in a subject by modulating (e.g., inhibiting) the expression of F-box only protein 7 (FBXO7), Eyes absent homology 2 (EYA2), or both in cancer cells to reduce AXL signaling. In some embodiments, the methods described herein may regulate epithelial-to-mesenchymal transition (EMT) and reduce growth, invasion, and metastasis of tumor. In some embodiments, the methods may further comprise administrating a therapeutically effective amount of a check-point inhibitor such as a programmed cell death protein 1 (PD-1) inhibitor.

Targeting EYA2 Tyr phosphatase activity can decrease mesenchymal phenotypes and enhance cancer cell immunogenicity, resulting in attenuated tumor growth and metastasis, increased infiltration of cytotoxic T and NK cells, and/or enhanced anti-PD-1 therapy response in tumors. In some aspects, EYA2 phosphatase inhibitors can act synergistically with anti-PD1 therapy to treat cancer by, for example, reducing the mesenchymal and/or immune evasion phenotypes of the cancer. As EYA2 acts downstream of SCF^{FBXW7}, FBXO7/EYA2 inhibitors can be effective in blocking immune escape pathways and immunotherapy resistance in, for example, F-boc WD-repeat containing protein (Fbxw7) mutant tumors. As such, in some aspects, disclosed herein is a method for treating immunotherapy resistance in tumors, more particularly in Fbxw7 mutant tumors, comprising administering a therapeutically effective amount of a FBXO7 inhibitor and/or a therapeutically effective amount EYA2 phosphatase inhibitor to a patient identified as having immunotherapy resistance and/or specifically as having Fbxw7 mutant tumors.

Also disclosed herein are methods for identifying an agent for treating or preventing a tumor growth in a patient having immune checkpoint blockade therapy resistant cancer. In some embodiments, the agent can be a small molecule, a peptide, an antibody, polynucleotides (e.g., DNA, RNA) or combination thereof. In some embodiments, the method comprises screening a compound library to identify compounds which bind to FBXO7, EYA2 for treating or preventing immune checkpoint blockade therapy resistant cancer.

### Definitions

As used herein, unless specified to the contrary, the following terms have the meaning indicated below.

As used herein, the term “comprise” or variations thereof such as “comprises” or “comprising” is to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone. It is further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure unless the context clearly dictates otherwise.

As used herein, the singular forms “a”, “an” and “the” include plural references unless the context clearly dictates otherwise. For example, the term “a sample” includes a plurality of samples, including mixtures thereof.

As used herein, the terms “determining,” “measuring,” “evaluating,” “assessing,” “assaying,” and “analyzing” are often used interchangeably to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the terms “treatment of” or “treating,” ‘applying”, or “palliating” or “ameliorating” are used interchangeably. These terms refer to an approach for obtaining beneficial or desired results including but not limited to therapeutic benefit and/or a prophylactic benefit. By “therapeutic benefit,” it is meant the eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease or condition, or to a patient reporting one or more of the physiological symptoms of a disease, even though a diagnosis of this disease has not been made.

As used herein, the terms “subject,” “individual,” or “patient” are often used interchangeably. A “subject” can be a biological entity containing expressed genetic materials. The biological entity can be a plant, animal, or microorganism, including, for example, bacteria, viruses, fungi, and protozoa. The biological entity can be an organ, a tissue, a cell, a plurality of cells, a cell population, or its progeny from an individual organism, containing multiple distinct biological entities within similar tissues, cells, and their progeny. The subject can be tissues, cells and their progeny of a biological entity obtained in vivo or cultured in vitro. The subject can be a mammal. The mammal can be a human. The subject can be diagnosed or suspected of being at high risk for a disease or any pathological condition. In some cases, the subject is not necessarily diagnosed or suspected of being at high risk for the disease.

The term “combination” embraces the administration of therapeutic agents (for example, a FBXO7 inhibitor or EYA2 phosphatase inhibitor) and an immunogenic composition (e.g., one or more checkpoint inhibitors), as part of a treatment regimen intended to provide a beneficial (additive or synergistic) effect from the co-action of one or more of these therapeutic agents. The combination may also include one or more additional agents, for example, but not limited to, chemotherapeutic agents, anti-angiogenesis agents and agents that reduce immune-suppression. The beneficial effect of the combination includes, but is not limited to, pharmacokinetic or pharmacodynamic co-action resulting from the combination of therapeutic agents. Administration of these therapeutic agents in combination typically is carried out over a defined time period (for example, minutes, hours, days, or weeks depending upon the combination selected).

“Combination therapy” is intended to embrace administration of the therapeutic agents disclosed herein in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present disclosure may comprise a FBXO7 inhibitor or EYA2 phosphatase inhibitor (for example, an FBXO7 siRNA molecule) and a checkpoint inhibitor administered at the same or different times, or they can be formulated as a single, co-formulated pharmaceutical composition comprising the two compounds. As another example, a combination of the present disclosure (e.g., a FBXO7 siRNA molecule and a checkpoint inhibitor anti-PD-1) may be formulated as separate pharmaceutical compositions that can be administered at the same or different time. As used herein, the term “simultaneously” is meant to refer to administration of one or more agents at the same time. For example, in certain embodiments, a neoplasia vaccine or immunogenic composition and a checkpoint inhibitor are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be affected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range. In certain instances, the term “about” also includes the exact number (i.e., about 5 includes 5).

As used herein, the terms “extracellular matrix” (ECM) or “matrix” refer to one or more substances that provide substantially the same conditions for supporting cell growth as provided by an extracellular matrix synthesized by feeder cells. The matrix can be provided on a substrate. Alternatively, the component(s) comprising the matrix can be provided in solution. The ECM thus encompasses essentially all secreted molecules that are immobilized outside of the cell. In vivo, the ECM provides order in the extracellular space and serves functions associated with establishing, separating, and maintaining differentiated tissues and organs. The ECM is a complex structure that is found, for example, in connective tissues and basement membranes, also referred to as the basal lamina. Connective tissue typically contains isolated cells surrounded by ECM that is naturally secreted by the cells. Components of the ECM have been shown to interact with and/or bind growth and differentiation factors, cytokines, matrix metalloproteases (MMPs), tissue inhibitors of metalloproteases (TIMPs), and other soluble factors that regulate cell proliferation, migration, and differentiation. Descriptions of the ECM and its components can be found in, among other places, Guidebook to the Extracellular Matrix, Anchor, and Adhesion Proteins, 2d ed., Kreis and Vale, eds., Oxford University Press, 1999 (“Kreis et al.”); Geiger et al., Nature Reviews Molecular Cell Biology 2:793-803, 2001; Iozzo, Annual Review of Biochemistry, 1998, Annual Reviews, Palo Alto, Calif.; Boudreau and Jones, Biochem. J. 339:481-88, 1999; Extracellular Matrix Protocols, Streuli and Grant, eds., Humana Press 2000; Metzler, Biochemistry the Chemical Reactions of Living Cells, 2d ed., vol. 1, 2001, Academic Press, San Diego, particularly chapter 8; and Lanza et al., particularly chapters 4 and 20.

As used herein, the terms “cancer” or “tumor” refers to various types of malignant neoplasms and tumors, including primary tumors, and tumor metastasis. Non-limiting examples of cancers which can be detected by the sensor array and system of the present disclosure are brain, ovarian, colon, prostate, kidney, bladder, breast, lung, oral, and skin cancers. Specific examples of cancers are: carcinomas, sarcomas, myelomas, leukemias, lymphomas and mixed type tumors. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

As used herein, the term “substantially undifferentiated” means that population of stem cells (e.g., primate primordial stem cells) contains at least about 50%, preferably at least about 60%, 70%, or 80%, and even more preferably, at least about 90%, undifferentiated, stem cells.

As used herein, the term “population” in reference to cells means two or more cells. A “homogeneous population” means a population comprising substantially only one cell type. A “cell type” can be cells of the same lineage or sub-type having substantially the same physiological status. Preferred homogeneous populations of the disclosure comprise substantially only early neuro-ectoderm-like Neural Precursor Cells (hNPCs). Reference to a “substantially pure homogeneous population of hNPCs” refers to a human neural precursor cell population in which a substantial number of the total population of the cells are of the same type and/or are in the same state of differentiation. Preferably, a “substantially pure homogeneous population” of neural cell precursor cells comprises a population of cells of which at least about 50% are of the same cell type, more preferably that at least about 75% are of the same cell type, even more preferably at least about 85% are of the same cell type, still even more preferably at least about 95% of the cells are the same type, and even more preferably at least about 97%, 98%, 99% or 100% are of the same cell type. In one embodiment, substantially homogeneous population of the disclosure is at least 99% of the same cell type. In another preferred embodiment, the preferred substantially homogeneous population of the disclosure is 100% of the same cell type. The substantially pure homogeneous population of hNPCs are generally obtained after about 10-12 days following the methods as disclosed herein.

As used herein, the term “activity” refers to protein biological or chemical function.

The section headings used herein are for organizational purposes only and are not to be construed as limiting the subject matter described.

### Methods

Provided herein are methods of treating cancer in a subject comprising administering a therapeutically effective amount of treatment to the subject, wherein the treatment comprises (i) an F-box only protein 7 (FBXO7) inhibitor, an Eyes absent homology 2 (EYA2) inhibitor, or both, (ii) a chemical entity that inhibits the interaction between FBXO7 and EYA2, or (iii) a chemical entity that interferes with the stabilization of FBXO7 and EYA2.

In some embodiments, the methods described herein may comprise treating cancer in a subject comprising administering a therapeutically effective amount of treatment to the subject, wherein the treatment comprises an F-box only protein 7 (FBXO7) inhibitor. In some embodiments, the methods described herein may comprise treating cancer in a subject comprising administering a therapeutically effective amount of treatment to the subject, wherein the treatment comprises an Eyes absent homology 2 (EYA2) inhibitor. In some embodiments, the methods described herein may comprise treating cancer in a subject comprising administering a therapeutically effective amount of treatment to the subject, wherein the treatment comprises an F-box only protein 7 (FBXO7) inhibitor, and an Eyes absent homology 2 (EYA2) inhibitor.

In some embodiments, the methods described herein may include administering to a subject a composition that inhibits FBXO7 expression or activity (referred to herein as FBXO7 inhibitor). The term “FBXO7” as used herein refers to human FBXO7, variants of human FBXO7, isoforms and species homologs, and analogs having at least one common epitope with FBXO7. In some cases, the FBXO7 inhibitor can be a nucleic acid-based inhibitor. In some cases, the nucleic acid can be DNA. In some cases, the FBXO7 inhibitor is an RNA-based inhibitor. In some embodiments, the FBXO7 inhibitor can be an siRNA. In some cases, the FBXO7 inhibitor can be a peptide, for example, an antibody. In other embodiments, the FBXO7 inhibitor can be a small molecule.

In some embodiments, in the methods described herein, the FBXO7 inhibitor may decrease the expression of EMT-associated genes (e.g., JUN, MYC, ZEB1, TUBA1C, CDK6, AKT3, and GAS6). In some embodiments, in the methods described herein, the FBXO7 inhibitor may downregulate EMT transcription factors ZEB1, ZEB2, and TWIST. In some embodiment, the FBXO7 inhibitor may increase immune stimulatory genes (e.g., IFNGR1, CXCL5, and MICA/MICB). In some embodiments, the FBXO7 inhibitor may upregulate immune stimulatory IFN-α, and IFN-β, chemokines CXCL9, CXCL10, HLA-A, and HLA-B.

In some embodiments, a FBXO7 inhibitor can reduce expression of FBXO7, EMT-associated genes, or transcription factors described herein by about 1.5 folds to about 50 folds. In some embodiments, a FBXO7 inhibitor can reduce expression of FBXO7, EMT-associated genes, or transcription factors described herein by about 1.5 folds to about 2 folds, about 1.5 folds to about 3 folds, about 1.5 folds to about 4 folds, about 1.5 folds to about 5 folds, about 1.5 folds to about 10 folds, about 1.5 folds to about 15 folds, about 1.5 folds to about 20 folds, about 1.5 folds to about 25 folds, about 1.5 folds to about 30 folds, about 1.5 folds to about 40 folds, about 1.5 folds to about 50 folds, about 2 folds to about 3 folds, about 2 folds to about 4 folds, about 2 folds to about 5 folds, about 2 folds to about 10 folds, about 2 folds to about 15 folds, about 2 folds to about 20 folds, about 2 folds to about 25 folds, about 2 folds to about 30 folds, about 2 folds to about 40 folds, about 2 folds to about 50 folds, about 3 folds to about 4 folds, about 3 folds to about 5 folds, about 3 folds to about 10 folds, about 3 folds to about 15 folds, about 3 folds to about 20 folds, about 3 folds to about 25 folds, about 3 folds to about 30 folds, about 3 folds to about 40 folds, about 3 folds to about 50 folds, about 4 folds to about 5 folds, about 4 folds to about 10 folds, about 4 folds to about 15 folds, about 4 folds to about 20 folds, about 4 folds to about 25 folds, about 4 folds to about 30 folds, about 4 folds to about 40 folds, about 4 folds to about 50 folds, about 5 folds to about 10 folds, about 5 folds to about 15 folds, about 5 folds to about 20 folds, about 5 folds to about 25 folds, about 5 folds to about 30 folds, about 5 folds to about 40 folds, about 5 folds to about 50 folds, about 10 folds to about 15 folds, about 10 folds to about 20 folds, about 10 folds to about 25 folds, about 10 folds to about 30 folds, about 10 folds to about 40 folds, about 10 folds to about 50 folds, about 15 folds to about 20 folds, about 15 folds to about 25 folds, about 15 folds to about 30 folds, about 15 folds to about 40 folds, about 15 folds to about 50 folds, about 20 folds to about 25 folds, about 20 folds to about 30 folds, about 20 folds to about 40 folds, about 20 folds to about 50 folds, about 25 folds to about 30 folds, about 25 folds to about 40 folds, about 25 folds to about 50 folds, about 30 folds to about 40 folds, about 30 folds to about 50 folds, or about 40 folds to about 50 folds. In some embodiments, a FBXO7 inhibitor can reduce expression of FBXO7, EMT-associated genes, or transcription factors described herein by about 1.5 folds, about 2 folds, about 3 folds, about 4 folds, about 5 folds, about 10 folds, about 15 folds, about 20 folds, about 25 folds, about 30 folds, about 40 folds, or about 50 folds. In some embodiments a FBXO7 inhibitor can reduce expression of FBXO7, EMT-associated genes, or transcription factors described herein by at least about 1.5 folds, about 2 folds, about 3 folds, about 4 folds, about 5 folds, about 10 folds, about 15 folds, about 20 folds, about 25 folds, about 30 folds, or about 40 folds. In some embodiments, a FBXO7 inhibitor can reduce expression of FBXO7, EMT-associated genes, or transcription factors described herein by at most about 2 folds, about 3 folds, about 4 folds, about 5 folds, about 10 folds, about 15 folds, about 20 folds, about 25 folds, about 30 folds, about 40 folds, or about 50 folds.

In some embodiments, a FBXO7 inhibitor can upregulate immune stimulatory IFN-α, and IFN-β, chemokines CXCL9, CXCL10, HLA-A, and HLA-B about 1.5 folds to about 50 folds. In some embodiments, a FBXO7 inhibitor can upregulate immune stimulatory IFN-α, and IFN-β, chemokines CXCL9, CXCL10, HLA-A, and HLA-B about 1.5 folds to about 2 folds, about 1.5 folds to about 3 folds, about 1.5 folds to about 4 folds, about 1.5 folds to about 5 folds, about 1.5 folds to about 10 folds, about 1.5 folds to about 15 folds, about 1.5 folds to about 20 folds, about 1.5 folds to about 25 folds, about 1.5 folds to about 30 folds, about 1.5 folds to about 40 folds, about 1.5 folds to about 50 folds, about 2 folds to about 3 folds, about 2 folds to about 4 folds, about 2 folds to about 5 folds, about 2 folds to about 10 folds, about 2 folds to about 15 folds, about 2 folds to about 20 folds, about 2 folds to about 25 folds, about 2 folds to about 30 folds, about 2 folds to about 40 folds, about 2 folds to about 50 folds, about 3 folds to about 4 folds, about 3 folds to about 5 folds, about 3 folds to about 10 folds, about 3 folds to about 15 folds, about 3 folds to about 20 folds, about 3 folds to about 25 folds, about 3 folds to about 30 folds, about 3 folds to about 40 folds, about 3 folds to about 50 folds, about 4 folds to about 5 folds, about 4 folds to about 10 folds, about 4 folds to about 15 folds, about 4 folds to about 20 folds, about 4 folds to about 25 folds, about 4 folds to about 30 folds, about 4 folds to about 40 folds, about 4 folds to about 50 folds, about 5 folds to about 10 folds, about 5 folds to about 15 folds, about 5 folds to about 20 folds, about 5 folds to about 25 folds, about 5 folds to about 30 folds, about 5 folds to about 40 folds, about 5 folds to about 50 folds, about 10 folds to about 15 folds, about 10 folds to about 20 folds, about 10 folds to about 25 folds, about 10 folds to about 30 folds, about 10 folds to about 40 folds, about 10 folds to about 50 folds, about 15 folds to about 20 folds, about 15 folds to about 25 folds, about 15 folds to about 30 folds, about 15 folds to about 40 folds, about 15 folds to about 50 folds, about 20 folds to about 25 folds, about 20 folds to about 30 folds, about 20 folds to about 40 folds, about 20 folds to about 50 folds, about 25 folds to about 30 folds, about 25 folds to about 40 folds, about 25 folds to about 50 folds, about 30 folds to about 40 folds, about 30 folds to about 50 folds, or about 40 folds to about 50 folds. In some embodiments, a FBXO7 inhibitor can upregulate immune stimulatory IFN-α, and IFN-β, chemokines CXCL9, CXCL10, HLA-A, and HLA-B about 1.5 folds, about 2 folds, about 3 folds, about 4 folds, about 5 folds, about 10 folds, about 15 folds, about 20 folds, about 25 folds, about 30 folds, about 40 folds, or about 50 folds. In some embodiments, a FBXO7 inhibitor can upregulate immune stimulatory IFN-α, and IFN-β, chemokines CXCL9, CXCL10, HLA-A, and HLA-B at least about 1.5 folds, about 2 folds, about 3 folds, about 4 folds, about 5 folds, about 10 folds, about 15 folds, about 20 folds, about 25 folds, about 30 folds, or about 40 folds. In some embodiments, a FBXO7 inhibitor can upregulate immune stimulatory IFN-α, and IFN-β, chemokines CXCL9, CXCL10, HLA-A, and HLA-B at most about 2 folds, about 3 folds, about 4 folds, about 5 folds, about 10 folds, about 15 folds, about 20 folds, about 25 folds, about 30 folds, about 40 folds, or about 50 folds.

In some embodiments, in the methods described herein, a FBXO7 inhibitor can decrease or prevent AXL by modulating EYA2/SIX1-GAS6-mediated activation of AXL signaling by interfering with the binding and stabilizing of FBXO7 to EYA2. In some embodiments, a FBXO7 inhibitor can modulate SKP1-Cullin-F-box (SCF)^{FBXW7} E3 ubiquitin ligase recognition of EYA2, thus promoting EYA2 degradation. In some embodiments, a FBXO7 inhibitor can prevent nuclear accumulation of EYA2. In some embodiments, the FBXO7 inhibitor may decrease EYA2 binding at various genomic regions, including promoters, exons, and introns. In some embodiments, the FBXO7 inhibitor may prevent biding of EYA2 and SIX1 to the Gas6 promoter. In some embodiments, the FBXO7 inhibitor may decrease total and secreted GAS6 protein. In some embodiments, the FBXO7 inhibitor may decrease total AXL and phospho-AXL^{Tyr702}. In some embodiments, the FBXO7 inhibitor may decrease AXL surface expression by decreasing phosphor-p38^{Thr180/Tyr182}, phospho-AKT^{T308}, and SOCS1 and SOCS3.

In some embodiments, in the methods described herein, the FBXO7 inhibitor may downregulate key pathways associated with cell movement, stem cell maintenance, and cell division in a cancer cell. In some embodiments, the FBXO7 inhibitor may upregulate key pathways associated with immune stimulatory pathways, antigen processing and presentation, cytokine secretion, and acute inflammatory response in a cancer cell. In some embodiments, the FBXO7 inhibitor may regulate a mesenchymal maintenance in a cancer cell. In some embodiments, the FBXO7 inhibitor may induce immune stimulatory phenotypes promoting susceptibility to adaptive immunity in a cancer cell.

In some embodiments, the methods described herein may include administering to a subject a composition that inhibits EYA2 expression or activity (referred to herein as EYA2 phosphatase inhibitor). The term “EYA2” as used herein refers to human EYA2, variants of human EYA2, isoforms and species homologs, and analogs having at least one common epitope with EYA2. In some embodiments, the compound can be a nucleic acid-based inhibitor. In some cases, the nucleic acid can be DNA. In some cases, the EYA2 phosphatase inhibitor is an RNA-based inhibitor. In some embodiments, the EYA2 phosphatase inhibitor can be an siRNA. In some cases, the EYA2 phosphatase inhibitor can be a peptide, for example, an antibody. In some embodiments, the EYA2 phosphatase inhibitor can be a small molecule or a phosphatase inhibitor.

Non-limiting example of small molecule EYA2 phosphatase inhibitor can include a compound:

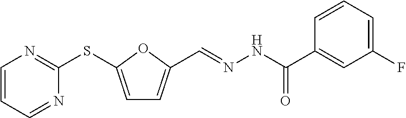 [SMILES: O=C(N/N=C/c1ccc(Sc2ncccn2)o1)c1cccc(F)c1]

In another non-limiting example of EYA2 phosphatase inhibitor can include a compound:

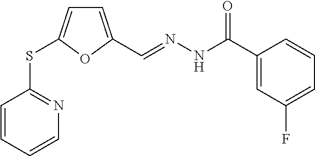 [SMILES: O=C(N/N=C/c1ccc(Sc2ccccn2)o1)c1cccc(F)c1]

In some embodiments, the EYA2 phosphatase inhibitor may decrease the total and secreted GAS6 protein, thereby decreasing or preventing AXL signaling in a cancer cell. In some embodiments, the EYA2 phosphatase inhibitor may downregulate key pathways associated with microtubule-based movement, microtube cytoskeleton organization, cell migration in hindbrain, stem cell proliferation, cell division, mitotic cell cycles in a cancer cell. In some embodiments, the EYA2 phosphatase inhibitor may upregulate key pathways associated with chemokine production, cytokine secretion, T-cell activation, inflammatory response, antigen processing and presenting, interferon gamma, and response to type I interferon beta in a cancer cell.

In some embodiments, provided herein are methods of suppressing FBXO7/EYA2-activation in a cancer cell comprising (i) contacting the cancer cell with an inhibitor of F-box only protein 7 (FBXO7), or (ii) contacting the cancer cell with an inhibitor of FBXO7 synthesis.

In some embodiments, the methods described herein may include a knockdown mechanism comprising at least one of an RNA interference (RNAi), a small interfering RNA (siRNA), a short hairpin RNA (shRNA), a hairpin siRNA, a precursor microRNA (pre-miRNA) or a microRNA (miRNA), a zinc finger nuclease (ZFN), a bacterial RNA-guided endonuclease, a TAL-effector nuclease (TALEN), or an antisense oligonucleotide (ASO) directed towards FBXO7, FBXO7 synthesis, EYA2, or a combination thereof.

In some embodiments, the methods described herein may include a bacterial RNA-guided endonuclease directed towards FBXO7, FBXO7 synthesis, EYA2, or a combination thereof, which comprise a Cas protein of a CRISPR/Cas system. In some embodiments, a Cas protein can be a Class 1 or a Class 2 Cas protein. In some embodiments, a Cas protein can be a Type I, Type II, Type III, Type IV, Type V, or Type VI Cas protein.

In some embodiments, a non-limiting examples of Cas proteins can include c2c1, C2c2, c2c3, Cas1, Cas1B, Cas2, Cas3, Cas4, Cas5, Case (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas1O, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cu1966, and homologs or modified versions thereof. In some embodiments, the Cas protein can be a deactivated Cas.

In some embodiments, a Cas protein can be from any suitable organism. In some embodiments, a suitable organism can comprise Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, an organism can comprise Streptococcus pyogenes (S. pyogenes).

In some embodiments a bacterial RNA-guided endonuclease directed towards FBXO7, FBXO7 synthesis, EYA2, or a combination thereof can be a fusion protein comprising a functional domain. In some embodiments, a bacterial RNA-guided endonuclease can be fused to a cleavage domain, an epigenetic modification domain, a transcriptional activation domain, or a transcriptional repressor domain. In some embodiments, a non-limiting example of a suitable fusion partner can include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, or any combination thereof. In some embodiments, the transcriptional repression domain may comprise a DNMT protein such as DNMT1, DNMT3A, DNMT3B, DNMT3L, a histone deacetylase (HDAC), a histone acetyltransferase (HAT), a histone methylase, or an enzyme that SUMOylates or biotinylates a histone and/or other enzyme domain that allows post-translation histone modification regulated gene repression. In some embodiments, a polynucleotide guided endonuclease can also be fused to a heterologous polypeptide providing increased or decreased stability. In some embodiments, a fused domain or heterologous polypeptide can be located at an N-terminus, a C-terminus, or internally within a polynucleotide guided endonuclease.

In some embodiments, a type II CRISPR/Cas system from bacteria can employ a crRNA and tracrRNA to guide a Cas polypeptide to a nucleic acid target (e.g., FBXO7). In some embodiments, a crRNA (CRISPR RNA) can contain a region complementary to one strand of a double strand DNA target (e.g., FBXO7 or EYA2). In some embodiments, a crRNA can base pair with a tracrRNA (trans-activating CRISPR RNA) to form an RNA duplex that can direct a Cas polypeptide to recognize and optionally cleave a DNA target.

In some embodiments, the inhibitor of FBXO7, FBXO7 synthesis, EYA2, or a combination thereof can be a CRISPR-Cas9 complex comprising a Cas9 protein or a polynucleotide encoding the Cas9 protein, and a guide RNA or polynucleotide encoding the guide RNA, wherein the guide RNA hybridizes with a nucleic acid sequence of FBXO7 or EYA2.

In some embodiments, compositions and methods of a disclosure can use Transcription activator-like effector nucleases (TALENs; TAL effector nucleases). In some embodiments, TALENs can be a class of sequence-specific nucleases. In some embodiments, TALENs can be used to cleave (e.g., double-strand breaks) at specific target sequences (e.g., in a genome of a plant or other organism). In some embodiments, TALENs can be created by fusing a native or engineered transcription activator-like (TAL) effector, or functional part thereof, to the catalytic domain of an endonuclease, such as, for example, FokI. In some embodiments, a unique, modular TAL effector DNA binding domain can allow for a design of proteins with potentially any given DNA recognition specificity.

Disclosed herein in some embodiments, are compositions and methods comprising use of zinc finger nucleases (ZFNs). In some embodiments, ZFNs can be engineered cleavage (e.g., double-strand break) inducing agents comprised of a zinc finger DNA binding domain and a double-strand-break-inducing agent domain. In some embodiments, recognition site specificity can be conferred by a zinc finger domain, which can comprise two, three, or four zinc fingers, for example having a C2H2 structure. In some embodiments, a Zinc finger domain can be amenable for designing polypeptides which specifically bind a selected polynucleotide recognition sequence. In some embodiments, a ZFN can consist of an engineered DNA-binding zinc finger domain linked to a non-specific endonuclease domain, for example, a nuclease domain from a Type IIS endonuclease such as FokI. In some embodiments, additional functionalities can be fused to a zinc-finger binding domain, including transcriptional activator domains, transcription repressor domains, and methylases. In some examples, a dimerization of nuclease domain can be required for cleavage activity. In some embodiments, each zinc finger can recognize, for example, three consecutive base pairs in a target DNA. In some embodiments, a 3-finger domain can recognize a sequence of 9 contiguous nucleotides, with a dimerization requirement of a nuclease, two sets of zinc finger triplets can be used to bind an 18-nucleotide recognition sequence.

In some embodiments, the method disclosed herein may comprise administering a therapeutically effective amount of treatment to the subject, wherein the treatment comprises a chemical entity that inhibits the interaction between FBXO7 and EYA2. In some embodiments, the methods disclosed herein can comprise administering a therapeutically effective amount of treatment to the subject, wherein the treatment comprises a chemical entity that interferes with the stabilization of EYA2 by FBXO7.

In some embodiments, a chemical entity can inhibit interaction between EYA2 and FBXO7 and/or stabilization of EYA2 by FBXO7 by interfering with binding of FBXO7 to a fragment of EYA2. In some embodiments, a chemical entity can inhibit binding of FBXO7 to specific regions of EYA2. In some embodiments, a chemical entity can inhibit binding of FBXO7 to transactivation domain of EYA2. In some embodiments, a chemical entity can inhibit binding of FBXO7 to SEQ ID NO:1. In some embodiments, a chemical entity can inhibit binding of FBXO7 to a fragment comprising amino acid 1-252 of SEQ ID NO:1.

```
(human EYA2)
SEQ ID No: 1
    1 MVELVISPSL TVNSDCLDKL KFNRADAAVW
        TLSDRQGITK SAPLRVSQLF
  51 SRSCPRVLPR QPSTAMAAYG QTQYSAGIQQ
        ATPYTAYPPP AQAYGIPSFS
101 IKTEDSLNHS PGQSGFLSYG SSFSTSPTGQ
        SPYTYQMHGT TGFYQGGNGL
151 GNAAGFGSVH QDYPSYPGFP QSQYPQYYGS
        SYNPPYVPAS SICPSPLSTS
201 TYVLQEASHN VPNQSSESLA GEYNTHNGPS
        TPAKEGDTDR PHRASDGKLR
251 GRSKRSSDPS PAGDNEIERV FVWDLDETII
        IFHSLLTGTF ASRYGKDTTT
301 SVRIGLMMEE MIFNLADTHL FENDLEDCDQ
        IHVDDVSSDD NGQDLSTYNF
351 SADGFHSSAP GANLCLGSGV HGGVDWMRKL
        AFRYRRVKEM YNTYKNNVGG
401 LIGTPKRETW LQLRAELEAL TDLWLTHSLK
        ALNLINSRPN CVNVLVTTTQ
451 LIPALAKVLL YGLGSVFPIE NIYSATKTGK
        ESCFERIMQR FGRKAVYVVI
501 GDGVEEEQGA KKHNMPFWRI SCHADLEALR
        HALELEYL
```

In some embodiments, a chemical entity of the present disclosure can have a binding activity to FBXO7 or EYA2 that is at least or equal or better than the binding activity observed between FBXO7 and EYA2. In some embodiments, a chemical entity of the present disclosure can have a binding activity or affinity to FBXO7 or EYA2 that is about 1-fold to about 15-fold greater than the binding activity observed between FBXO7 and EYA2. In some embodiments, a chemical entity of the present disclosure can have a binding activity or affinity to FBXO7 or EYA2 that is about 1-fold to about 2-fold, about 1-fold to about 3-fold, about 1-fold to about 4-fold, about 1-fold to about 5-fold, about 1-fold to about 6-fold, about 1-fold to about 7-fold, about 1-fold to about 8-fold, about 1-fold to about 9-fold, about 1-fold to about 10-fold, about 1-fold to about 15-fold, about 2-fold to about 3-fold, about 2-fold to about 4-fold, about 2-fold to about 5-fold, about 2-fold to about 6-fold, about 2-fold to about 7-fold, about 2-fold to about 8-fold, about 2-fold to about 9-fold, about 2-fold to about 10-fold, about 2-fold to about 15-fold, about 3-fold to about 4-fold, about 3-fold to about 5-fold, about 3-fold to about 6-fold, about 3-fold to about 7-fold, about 3-fold to about 8-fold, about 3-fold to about 9-fold, about 3-fold to about 10-fold, about 3-fold to about 15-fold, about 4-fold to about 5-fold, about 4-fold to about 6-fold, about 4-fold to about 7-fold, about 4-fold to about 8-fold, about 4-fold to about 9-fold, about 4-fold to about 10-fold, about 4-fold to about 15-fold, about 5-fold to about 6-fold, about 5-fold to about 7-fold, about 5-fold to about 8-fold, about 5-fold to about 9-fold, about 5-fold to about 10-fold, about 5-fold to about 15-fold, about 6-fold to about 7-fold, about 6-fold to about 8-fold, about 6-fold to about 9-fold, about 6-fold to about 10-fold, about 6-fold to about 15-fold, about 7-fold to about 8-fold, about 7-fold to about 9-fold, about 7-fold to about 10-fold, about 7-fold to about 15-fold, about 8-fold to about 9-fold, about 8-fold to about 10-fold, about 8-fold to about 15-fold, about 9-fold to about 10-fold, about 9-fold to about 15-fold, or about 10-fold to about 15-fold greater than the binding activity observed between FBXO7 and EYA2. In some embodiments, a chemical entity of the present disclosure can have a binding activity or affinity to FBXO7 or EYA2 that is about 1-fold, about 2-fold, about 3-fold, about 4-fold, about 5-fold, about 6-fold, about 7-fold, about 8-fold, about 9-fold, about 10-fold, or about 15-fold greater than the binding activity observed between FBXO7 and EYA2. In some embodiments, a chemical entity of the present disclosure can have a binding activity or affinity to FBXO7 or EYA2 that is at least about 1-fold, about 2-fold, about 3-fold, about 4-fold, about 5-fold, about 6-fold, about 7-fold, about 8-fold, about 9-fold, or about 10-fold greater than the binding activity observed between FBXO7 and EYA2. In some embodiments, a chemical entity of the present disclosure can have a binding activity or affinity to FBXO7 or EYA2 that is at most about 2-fold, about 3-fold, about 4-fold, about 5-fold, about 6-fold, about 7-fold, about 8-fold, about 9-fold, about 10-fold, or about 15-fold greater than the binding activity observed between FBXO7 and EYA2. In some embodiments, a chemical entity of the present disclosure has a binding activity or affinity to FBXO7 or EYA2 of at least about 1×10⁶, 1×10⁷, 1×10⁸ or 1×10⁹ M. In some embodiments, a chemical entity of the present disclosure is capable of inhibiting, inhibit or compete with FBXO7 or EYA2 either in vivo, in vitro or both.

In some embodiments, a chemical entity can interfere with the stabilization of EYA2 by FBXO7 by enhancing EYA2 K-48-linked polyubiquitylation by SCF^{FBXOW7}. In some embodiments, a chemical entity can enhance binding of SCF^{FBXOW7} to EYA2's a conserved CDC4 phosphodegron (CPD). In some embodiments, a chemical entity can interfere with the stabilization of EYA2 by FBXO7 by enhancing GSK3β-mediated phosphorylation of the substrate CPD.

In some embodiments, the method disclosed herein may further comprise administering (i) a FBXO7 inhibitor, a EYA2 phosphatase inhibitor or both, (ii) a chemical entity that inhibits the interaction between FBXO7 and EYA2, or (iii) a chemical entity that interferes with the stabilization of EYA2 by FBXO7, and combined with a therapeutic antibody (e.g., checkpoint inhibitor), a chemotherapeutic agent, an immunotherapeutic agent, a radiotherapeutic agent, an anti-neoplastic agent, an anti-cancer agent, an anti-fibrotic agent, an anti-angiogenic agent or, or any combination thereof.

In some embodiments, the therapeutic antibody may comprise a checkpoint inhibitor such as a PD-1 inhibitor (e.g., anti-PD-1) or an antigen-binding portion thereof, a PD-L1 inhibitor (e.g., anti-PD-L1) or an antigen-binding portion thereof. In some embodiments, a CTLA-4 inhibitor (e.g., anti-CTLA-4) or an antigen-binding portion thereof. In some cases, the inhibitors can be directed to other immune check points, for example, lymphocyte activation gene 3 LAG 3, T-cell membrane protein 3, and V-domain immunoglobulin suppressor of T-cell activation (VISTA). In some embodiments, the inhibitors can be directed against TIGIT signal transduction pathway. In some embodiments, the inhibitors can be directed against the ligands for TIGIT signaling. In some cases, the inhibitor can be directed against BTLA signaling.

In some embodiments, the present disclosure comprises administering to a subject suffering from cancer a composition comprising a therapeutically effective amount of a PD-1 inhibitor (e.g., anti-PD-1) or an antigen-binding portion thereof, in combination with a FBXO7 inhibitor, an EYA2 phosphatase inhibitor, or both. In certain embodiments, the inhibitor is the Ab or antigen-binding portion thereof is an IgG1 or IgG4 isotype. In other embodiments, the Ab or antigen-binding portion thereof is a mAb or antigen-binding portion thereof. The anti-PD-1 Ab used in the present method can be any therapeutic anti-PD-1 Ab of the present disclosure. In a preferred embodiment, the anti-PD-1 Ab is a mAb, which can be a chimeric, humanized or human Ab. In certain embodiments, the anti-PD-1 Ab is a CDR1 in the heavy chain variable region of 17D8, 2D3, 4H1, 5C4 (nivolumab), 4A11, 7D3 or 5F4, as described and clearly defined in U.S. Pat. No. 8,008,449, It includes the CDR2 and CDR3 domains and includes the CDR1, CDR2 and CDR3 domains within the light chain variable region. In a further embodiment, the anti-PD-1 Ab comprises heavy and light chain variable regions of 17D8, 2D3, 4H1, 5C4 (nivolumab), 4A11, 7D3 or 5F4, respectively. In a further embodiment, the anti-PD-1 Ab is 17D8, 2D3, 4H1, 5C4 (nivolumab), 4A11, 7D3 or 5F4. In a preferred embodiment, the anti-PD-1 Ab is nivolumab. Other antibody can be used in this method. In some embodiments, a mouse monoclonal antibody for human PD-1 is used in the method. For example, In vivo mAb anti PD-1 (BioXCell, Clone: RMP1-14, catalog number BE0146). Anti-PD-1 antibodies have been shown to be effective in human therapy, e.g., pembrolizumab (KEYTRUDA®, Merck Sharp & Dohme Corp.), which is an anti-PD-1 antibody approved for use in human therapy). In some cases, the anti-PD-1 antibody can be cemiplimab. In some cases, the anti-PD-1 antibody can be nibolumab (Opdivo®, Bristor-Myers Squibb). Additional PD-1 antibodies, such as Pidirisumab (CT-011) (CureTech Ltd.), are under clinical development. In some cases, the anti-PD-L1 antibody can be selected from atezolizumab, durvalumab, or avelumab. In some embodiments, a combination therapy comprising FBXO7 inhibitor, EYA2 phosphatase inhibitor and/or anti-PD-1 described herein can provide a more than an additive treatment effects on the subject's cancer than the administration of the therapeutically effective amount of FBXO7 inhibitor, EYA2 phosphatase inhibitor or anti-PD-1 alone.

In some embodiments, the present disclosure comprises administering to a subject suffering from cancer a composition comprising a chemotherapeutic agent in combination with a FBXO7 inhibitor, an EYA2 phosphatase inhibitor, or both. In some embodiments, the chemotherapeutic agents can be administered to a patient based on the need. The chemotherapeutic agents can be categorized by their mechanism of action in following groups: anti-metabolites/anti-cancer agents, such as pyrimidine analogs (floxuridine, capecitabine, and cytarabine); purine analogs, folate antagonists and related inhibitors, antiproliferative/antimitotic agents including natural products such as vinca alkaloid (vinblastine, vincristine) and microtubule such as taxane (paclitaxel, docetaxel), vinblastin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan, camptothesin), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) comprises any non-proteinaceous (i.e., non-peptidic) chemical compound useful in the treatment of cancer. Examples of chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiI1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK®; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL® and docetaxel (TAXOTERE®); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; FOLFIRI (fluorouracil, leucovorin, and irinotecan) and pharmaceutically acceptable salts, acids or derivatives of any of the above. One or more chemotherapeutic agent is used or included in the present application. For example, gemcitabine, nab-paclitaxel, and gemcitabine/nab-paclitaxel are used with the JAK inhibitor and/or PI3Kδ inhibitor for treating hyperproliferative disorders.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

The anti-angiogenic agents may include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inhibitor-2, cartilage-derived inhibitor, paclitaxel (nab-paclitaxel), platelet factor 4, protamine sulphate (clupeine), sulfated chitin derivatives (prepared from queen crab shells), sulfated polysaccharide peptidoglycan complex (sp-pg), staurosporine, modulators of matrix metabolism, including for example, proline analogs ((1-azetidine-2-carboxylic acid (LACA), cishydroxyproline, D,L-3,4-dehydroproline, thiaproline, .alpha.-dipyridyl, beta-aminopropionitrile fumarate, 4-propyl-5-(4-pyridinyl)-2(3H)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents may include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. Nos. 5,021,456; 5,5059,714; 5,120,764; 5,182,297; 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

In some embodiments, the methods described herein can be of use for therapy or treatment of cancer. Exemplary types of cancers that can be targeted include pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer, acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer.

In some embodiments, the cancer may comprise a solid tumor, such as adenocarcinoma, colon adenocarcinoma, rectal adenocarcinoma, pancreatic adenocarcinoma, glioblastoma, neuroendocrine tumors, colorectal carcinoma, gastric carcinoma, esophogeal carcinoma, neuroblastoma, or osteosarcoma.

In some embodiments, the solid tumor may comprise Fbxw7 mutant tumor. Non-limiting Fbxw7 mutant tumor type includes breast cancer, bladder cancer, cholangiocarcinoma, colon cancer, esophagus, leukemia, liver cancer, lung cancer, melanoma, bone cancer, ovarian cancer, prostate cancer, pancreas cancer, stomach cancer, T-cell acute lymphocytic leukemia, endometrial cancer, and gastric cancer.

In some embodiments, the methods described herein can reduce immunotherapy resistance. In some embodiments, the method described herein can prevent metastasis progression. In some embodiments, the method described herein can target metastatic tumor in a subject.

The method described herein can involve contacting a test agent with a FBXO7 or EYA2 that is overexpressed in cancer cells resistant to immune checkpoint blockade therapy and determining whether the test agent interacts with FBXO7 or EYA2. In some cases, the test agent can inhibit the interaction between FBXO7 and EYA2. In some cases, the test agent can destabilize EYA2. In some embodiments, the test agent can be a small molecule, a peptide, an antibody, polynucleotides (e.g., DNA, RNA) or combination thereof. In some embodiments, high throughput screening methods comprising a combinatorial small organic molecule or peptide library containing a large number of potential therapeutic compounds (potential modulator or ligand compounds) can be used for test agents.

In some embodiments, the interaction between the test agent and FBXO7 or EYA2 can be determined using a reporter protein (e.g., FBXO7 operably linked to a green fluorescent protein) by using fluorescence-based binding assay, fluorescence polarization assay, fluorescence resonance energy transfer assay, biomolecular fluorescence complementation assay or bioluminescence resonance energy transfer assay. In this scenario, the GFP signal can be used to determine whether the test agent interacts with FBXO7 or EYA2, where an increasing dose of the test agent correlates with a decrease in GFP signal. In some embodiments, other reporter protein such as biotin, avidin, streptavidin, neutravidin, the Fc region of an immunoglobulin, and the like can be used. In some embodiments, the test agent (e.g., small molecules) that modulates FBXO7 or EYA2 activity can also be determined by computer assisted drug design, in which a computer system is used to generate output value based on binding (e.g., the highest output value can be considered to have the most potential for further development as a therapeutic agent). The lead compounds can be selected that are at least one standard deviation from the mean. In some cases, the lead compounds can be selected that are at least 0.5 stand deviation from the mean. In some embodiments, screening a compound library may comprise performing molecular dynamic simulation on the crystal structure of FBXO7 or EYA2 using software such as GROMACS, AMBER, or CHARMM, to predict the protein's conformational dynamics and to generate an ensemble of protein structures. The simulation can be carried out under physiological conditions, such as pH and temperature, and for a sufficient duration, typically in the range of microseconds to milliseconds, to sample the conformational space of protein.

In some embodiments, the test agent that interacts with FBXO7 or EYA2 can be subjected to an in vitro, ex vivo, or in vitro test. The in vitro test can be a cell-based assay that measures cell proliferation or self-renewal capacity, such as extreme limiting dilution assay (ELDAs), or enzyme-linked immunosorbent assay (ELISA. Candidate test agents can be assayed initially in vitro for their ability to inhibit cell growth (i.e., their cytotoxicity, proliferation, or self-renewal capacity) using standard techniques. Assays that measure metabolic activity (such as tetrazolium-based assays) can also be used to assess the effect of test agents on cell activation and/or proliferation, due the fact that proliferating cells are metabolically more active than resting cells.

A variety of cancer cell-lines suitable for testing the test agents can be used according to the present disclosure. In some embodiments, in vitro testing of the test agents is conducted in a human cancer cell-line. Examples of suitable human cancer cell-lines for in vitro testing of the compounds of the present disclosure include, but are not limited to, colon and colorectal carcinoma cell lines such as HT-29, CaCo, LoVo, COLO320 and HCT-116; non-small cell lung cancer cell lines such as NCI-H460, small cell lung cancer cell lines such as H209; breast cancer cell lines such as MCF-7, T47D and MDA-MB-231; ovarian cancer cell lines such as SK-OV-3; prostate cancer cell lines such as PC-3 and DU-145; chronic myeloid leukemia cell lines such as K562; bladder cancer cell lines such as T24; brain cancer cell lines such as U-87-MG; pancreatic cancer cell lines such as AsPC-1, SU.86.86 and BxPC-3; kidney cancer cell lines such as A498 and Caki-1; liver cancer cell lines such as HepG2, and skin cancer cell lines such as A2058 and C8161. In some embodiments, the cancer cell can be resistant to immune checkpoint blockade therapy. In some embodiments, the cancer cell can be Fbxw7 mutant cells.

The selectivity of test agents may also be tested, i.e., the ability of the test agent to demonstrate some level of selective action toward cancer cells in comparison to normal proliferating cells. For example, the comparison of IC90 values, i.e., the molar concentration of a test agent required to cause 90% growth inhibition of exponentially growing cells, and the IC90 values for test agent can be evaluated in various cancer cell lines (such as those outlined above) and normal cells and test agents.

Toxicity of the test agents can also be initially assessed in vitro using standard techniques. For example, human primary fibroblasts can be treated in vitro with a test agent and then tested at different time points following treatment for their viability using a standard viability assay, such as the assays described above or the trypan-blue exclusion assay. Cells can also be assayed for their ability to synthesize DNA, for example, using a thymidine incorporation assay, and for changes in cell cycle dynamics, for example, using a standard cell sorting assay in conjunction with a fluorescence-activated cell sorting (FACS).

In some embodiments, the in vivo test can be a test in an animal model of immune checkpoint blockade therapy resistant cancer. The ability of the test agents to inhibit tumor growth, proliferation and/or metastasis in vivo can be determined in an appropriate animal model using standard techniques including xenograft models, in which a human tumor has been implanted into an animal.

For example, the test agent can be tested in vivo on solid tumor using mice that are subcutaneously grafted or injected with tumor fragment and monitor key parameters such as tumor growth. In some embodiments, in vivo test may include testing the effects of the test agents on tumor growth, differentiation, apoptosis, angiogenesis and/or tumor metastasis. In some embodiments, in vivo test may include orthopedic implantation of tumor into animals to assess the effect of the test agents on tumor growth and proliferation.

The ability of the test agents to act in combination with, or to sensitize a tumor to the effects of, another chemotherapeutic agent can also be tested in the above models. In this case, the test animals would be treated with both the chemotherapeutic agent and the candidate test agent. In vivo toxic effects of the test agents can be evaluated by standard techniques, for example, by measuring their effect on animal body weight during treatment and by performing hematological profiles and liver enzyme analysis after the animal has been sacrificed (survival assays). In some embodiments, the ex vivo test may include tumor sphere formation assay using a tissue sample obtained from a patient with immune checkpoint blockade therapy resistant cancer.

In some embodiments, an in vivo, in vitro, and ex vivo test may include determining whether the test agent interact with FBXO7 by measuring the levels of EMT-associated genes (e.g., JUN, MYC, ZEB1, TUBA1C, CDK6, AKT3, and GAS6), EMT transcription factors ZEB1, ZEB2, and TWIST, immune stimulatory genes (e.g., IFNGR1, CXCL5, and MICA/MICB), or immune stimulatory IFN-α, and IFN-β, chemokines CXCL9, CXCL10, HLA-A, and HLA-B.

### Pharmaceutical Composition

Compositions comprising (i) FBXO7 inhibitor, EYA2 phosphatase inhibitor or both, (ii) a chemical entity that inhibit the interaction between FBXO7 and EYA2, (iii) a chemical entity that interferes with the stabilization of EYA2 by FBXO7 described herein may comprise a pharmaceutical composition, for example a pharmaceutical composition containing a small molecule inhibitor or combination of small molecule inhibitors, one Ab or combination of Abs, or a nucleic acid capable of binding to an antigen (e.g., siRNA against FBX07), and a pharmaceutically acceptable carrier. As used herein, “pharmaceutically acceptable carrier” includes all solvents, dispersion media, coatings, antibacterial and antifungal agents, isotonic and absorption delaying agents, and other agents that are physiologically compatible. Preferably, the carrier is suitable for intravenous, intramuscular, subcutaneous, parenteral, spinal or epidermal administration (e.g., by injection or infusion). Pharmaceutical compositions of the present disclosure may comprise one or more pharmaceutically acceptable salts, antioxidants, aqueous and non-aqueous carriers, and/or adjuvants such as preservatives, wetting agents, emulsifying agents and dispersing agents. In addition, the compound may form a solvate with water or a common organic solvent. Such solvates are also envisioned.

The pharmaceutical compositions of the present disclosure comprising the inhibitors described herein as active ingredients may comprise a pharmaceutically acceptable carrier or additive, depending on the route of administration. Examples of such carriers or additives include water, pharmaceutically acceptable organic solvents, collagen, polyvinyl alcohol, polyvinylpyrrolidone, carboxyvinyl polymers, sodium carboxymethyl cellulose, sodium polyacrylate, sodium alginate, water-soluble dextran, carboxy. Methylstarch sodium, pectin, methylcellulose, ethylcellulose, xanthan gum, arabic gum, casein, gelatin, agar, diglycerin, glycerin, propylene glycol, polyethylene glycol, vaseline, paraffin, stearyl alcohol, stearic acid, human serum albumin (HSA), mannitol, Sorbitol, lactose, pharmaceutically acceptable surfactants and the like. The additives used are selected from, but not limited to, those described above or combinations thereof, as appropriate, depending on the dosage form of the present disclosure.

The formulation of the pharmaceutical composition varies depending on the route of administration selected (e.g., solution, emulsion). Suitable compositions comprising the inhibitors to be administered, e.g., siRNA against FBXO7, can be prepared in a physiologically acceptable vehicle or carrier. With respect to solutions or emulsions, suitable carriers include, for example, aqueous or alcoholic/aqueous solutions, emulsions, or suspensions, including saline and buffering media. Parenteral vehicles may include sodium chloride solution, ringer dextrose, dextrose and sodium chloride, lactated ringer, or fixed oil. Intravenous vehicles can include various additives, preservatives, or fluids, nutrients, or electrolyte supplements.

Various aqueous carriers such as sterile phosphate buffered saline, bacteriostatic water, water, buffered water, 0.4% saline, 0.3% glycine, etc., and albumin, can be subject to mild chemical modification. Other proteins can be included to enhance stability, such as proteins, globulin.

The therapeutic formulation of the inhibitors disclosed herein, for example, a FBXO4 inhibitor or a checkpoint inhibitor can be prepared by mixing the inhibitor with the desired degree of purity in the form of a lyophilized formulation or aqueous solution with an optional physiologically acceptable carrier, excipient, or stabilizer (see, for example, Remington's Pharmaceutical Sciences 16th edition, Osol, A. Ed. (1980)), and prepared for storage. Acceptable carriers, excipients, or stabilizers are non-toxic to the recipient at the dosages and concentrations employed, and include saline and/or buffers such as phosphoric acid, citric acid, and other organic acids; antioxidants including ascorbic acid and methionine; preservatives (such as, octadecyldimethylbenzylammonium chloride; hexamethonium chloride; benzalkonium chloride, benzethonium chloride; phenol, butyl or benzyl alcohol; alkylparabens such as methyl or propylparaben; catechol; resorcinol; cyclohexanol; 3-Pentanol; and m-cresol, etc.); low molecular weight (less than about 10 residues) polypeptides; proteins, such as serum albumin, gelatin, or immunoglobulin; hydrophilic polymers such as polyvinylpyrrolidone; amino acids such as glycin, glutamine, asparagine, histidine, arginine, or lysine; monosaccharides, disaccharides, and other carbohydrates including glucose, mannose, or dextrin; chelating agents such as EDTA; sugars such as sucrose, mannitol, trehalose, or sorbitol; salt-forming counterions such as sodium; metal complexes (e.g., Zn-protein complexes); and/or nonionic surfactants such as TWEEN™, PLURONICS™, or polyethylene glycol (PEG).

The formulations herein may also include two or more active compounds (for example, an anti-PD-1 and an inhibitor of FBXO7, EYA2 or both) preferably those having complementary activities that do not adversely affect each other, depending on the needs of the particular indication being treated. Such molecules are preferably present in combination in an amount effective for the intended purpose.

The active ingredient can be in a colloidal drug delivery system (e.g., in a colloidal drug delivery system, for example, in microcapsules prepared by, for example, coacervation techniques or by interfacial polymerization, e.g., hydroxyethyl cellulose or gelatin microcapsules and poly-(methylmethacrylate) microcapsules, respectively. Liposomes, albumin microspheres, microemulsions, nanoparticles, and nanocapsules), or can also be encapsulated in macroemulsions. Such techniques are described in Remington's Pharmaceutical Sciences 16th edition, Osol, A. et al. Ed. (1980).

The formulation used for in vivo administration can be made sterile by filtration through sterile filtration membranes. Aqueous suspensions may contain active compounds mixed with excipients suitable for making aqueous suspensions. Such excipients are suspending agents such as sodium carboxymethyl cellulose, methyl cellulose, hydroxypropyl methyl cellulose, sodium alginate, polyvinylpyrrolidone, tragacant gum and acacia gum. Dispersants or wetting agents can be naturally occurring phospholipids such as, for example. Recitin, or a condensate of an alkylene oxide with a fatty acid, such as polyoxyethylene stearate, or a condensate of an ethylene oxide with a long-chain aliphatic alcohol, such as heptadecaethyl-eneoxycetanol, or polyoxyethylene sorbitol monoole. It can be a condensate of an ethylene oxide such as ate with a partial ester obtained from a fatty acid and hexitol, or a condensate of an ethylene oxide with a partial ester obtained from a fatty acid and hexitol anhydride, such as polyethylene sorbitan monooleate. Aqueous suspensions may also contain one or more preservatives, such as ethyl, or n-propyl, p-hydroxybenzoate.

The chemical entities (e.g., antibodies or inhibitors) described herein can be lyophilized for storage and reconstituted with a suitable carrier prior to use. For example, PD-1 antibodies and FBXO7 antibodies described herein can be prepared and administered as a co-formation. In one aspect, at least two of the antibodies recognize and bind to different antigens. In another embodiment, at least two of the antibodies can specifically recognize and bind to different epitopes of the same antigen.

Any suitable lyophilization and reconstruction techniques can be employed. Lyophilization and reconstitution may result in varying degrees of loss of antibody activity and may have to be supplemented by adjusting the level of use. Dispersible powders and granules suitable for the preparation of aqueous suspensions by the addition of water can result in active compounds mixed with dispersants or wetting agents, suspending agents, and one or more preservatives. Suitable dispersants or wetting agents and suspending agents are exemplified by those already described above.

The concentration of the inhibitors (e.g., PD-1 antibody or FBXO7 antibody) in these formulations may vary widely, for example, from less than about 0.5% by weight to about 20% weight, usually from about 1% by weight or at least about 1% by weight to about 15 or 20% by weight. The concentration of the inhibitor is selected based on the liquid volume, viscosity, etc. according to the specific administration mode to be applied. Therefore, a typical pharmaceutical composition for parenteral injection can be configured to contain 1 mL sterile buffered water and 50 mg antibody. A typical composition for intravenous infusion can be configured to include 250 mL of sterile Ringer's solution and 150 mg of antibody. Practical methods for preparing compositions for parenteral administration can be found at, e.g., Remington's Pharmaceutical Science, 15th ed., Mack Publishing Company, Easton, Pa. The effective dosage of the antibody is in the range of from about 0.01 mg to about 1000 mg per kg body weight per dose.

The pharmaceutical composition can be in the form of a sterile injectable aqueous, oily suspension, dispersion, or sterile powder for an immediate preparation of a sterile injectable solution or dispersion. Suspensions can be formulated according to protocols using suitable dispersants or wetting agents and suspending agents described above. The sterile injectable preparation can be a sterile injectable solution or suspension of a non-toxic parenterally acceptable diluent or solvent such as, for example, 1,3-butanediol. The carrier can be a solvent or dispersion medium containing, for example, water, ethanol, polyol (e.g., glycerol, propylene glycol, liquid polyethylene glycol, etc.), a suitable mixture thereof, vegetable oil, Ringer's solution, and isotonic sodium chloride solution. In addition, sterile fixed oils have traditionally been adopted as solvents or suspension media. For this purpose, any non-irritating fixed oil can be employed, including synthetic mono- or diglycerides. In addition, fatty acids such as oleic acid are used in the preparation of injections.

In all cases, the form must be sterile and fluid enough to be easily injected. Appropriate fluidity can be maintained, for example, by the use of coating agents such as lecithin, by the maintenance of the required particle size in the case of dispersion, and by the use of surfactants. It must be stable under manufacturing and storage conditions and must be preserved against the contaminating effects of microorganisms such as bacteria and fungi. Blocking the action of microorganisms can be provided by various antibacterial and antifungal agents such as parabens, chlorobutanol, phenols, sorbic acid, thimerosal and the like. In many cases it is desirable to include isotonic agents such as sugar or sodium chloride. Long-term absorption of the injectable composition can be provided by using an agent that delays absorption in the composition, such as aluminum monostearate or gelatin.

Compositions useful for administration can be formulated with uptake or absorption enhancers to increase their effectiveness. Such enhancers include, for example, salicylate, glycocholate/linoleate, glycolate, aprotinin, bacitracin, SDS, caplate and the like. See, for example, Fix (J. Pharma. Sci., 85: 1282-1285 (1996)) and Oliyai and Stella (Ann. Rev. Pharmacol. Toxicol., 32: 521-544 (1993)).

A composition described herein can be intended for use in inhibiting target activity, including binding of a target to its cognate receptor or ligand, target-mediated signaling, and the like. For example, a composition described herein can be intended for use in inhibiting FBXO7 activity and/or PD-1 antibody. In particular, the compositions can exhibit inhibitory properties at concentrations that are substantially free of side effects and are therefore useful for long-term therapeutic protocols. For example, co-administration of an antibody composition with another more toxic cytotoxic drug can effectively reduce the toxic side effects of a patient while achieving beneficial inhibition of the condition or disorder being treated.

In addition, the hydrophilic and hydrophobic properties of the compositions intended for use in the present disclosure can be designed to be balanced, thereby enhancing their availability for both in vitro and especially in vivo use, but such a balance. In some embodiments, the compositions intended for use in the present disclosure can have appropriate solubility in an aqueous medium that allows absorption and bioavailability in the body, and the compound has a putative effect across the cell membrane. It also has a certain solubility in the lipids that allow it to enter the site. Therefore, the expected effectiveness of antibody compositions is maximized when they can be delivered to the target antigen active site,

### Administration and Dosing

In one aspect, the methods of the present disclosure include the steps of administering a pharmaceutical composition.

The compositions comprising FBXO7 inhibitor and/or EYA2 phosphatase inhibitors described herein can be administered by any suitable means, including parenteral, subcutaneous, intraperitoneal, intrapulmonary, and intranasal, and if topical treatment is desired, intralesional administration. Parenteral injections include intravenous, intraarterial, intraperitoneal, intramuscular, intradermal, or subcutaneous administration. In addition, the compositions described herein (e.g., FBXO7 or EYA2 phosphatase inhibitor) can be administered by pulse injection, when used at reduced doses. Other methods of administration are also envisioned, including topical, particularly transdermal, transmucosal, rectal, oral, or topical administration, e.g., through a catheter placed near the desired site. When used in conjunction with one or more chemotherapeutic agents, the FBXO7 inhibitor and/or EYA2 phosphatase inhibitor can be administered prior to, or after, administration of the chemotherapeutic agents, or they can be administered concomitantly. The one or more chemotherapeutic may also be administered systemically, for example, by bolus injection, continuous infusion, or oral administration.

In one embodiment, administration can be directed to the site of the cancer or to the affected tissue in need of treatment, either by direct injection into a site where the formulation can be delivered internally, or through a sustained delivery or sustained release mechanism. For example, a biodegradable microsphere or capsule or other biodegradable polymer composition (e.g., soluble polypeptide, antibody, or small molecule) capable of sustained delivery of the composition is implanted near or to the site of the cancer can be included in the formulations of the present disclosure.

The dosing regimen is adjusted to provide the optimal desired response, e.g., a therapeutic response or minimal side effects. For example, the daily dosages of the FBXO7 and/or EYA2 phosphatase inhibitors of the present disclosure typically falls within the range of about 0.01 to about 100 mg/kg of body weight, in single or divided dose. Examples of ranges for the inhibitors in each dosage unit are from about 0.05 to about 100 mg, or more usually, from about 1.0 to about 50 mg. In some cases, exemplary treatment regimens can be once per week, once every two weeks, once every three weeks, once every four weeks, once every month, once every three months, or once every three to six months. Dosage and scheduling can vary during treatment. For example, the dosing schedule can be such that the pharmaceutical compositions (e.g., comprising FBXO7 siRNA) is (i) administered every 2 weeks on a 6-week cycle; (ii) administered 6 times every 4 weeks, then every 3 months; (iii) administered every 3 weeks; (iv) 3 to 10 mg/kg (body weight) is administered once, and then 1 mg/kg (body weight) is administered every 2 to 3 weeks. In other cases, given that IgG4 Abs typically have a half-life of 2-3 weeks, the preferred dosing regimen for anti-PD-1 or anti-FBXO7 Abs of the present disclosure can be via intravenous administration, from 0.3 to 10 mg/kg (body weight), preferably 3 to 10 mg/kg (body weight), more preferably 3 mg/kg (body weight), and such Abs are up to a cycle of 6 or 12 weeks until complete response or confirmation of progressive disease. It is administered every 14 days.

Administration of multiple agents, including, but not limited to, chemotherapeutic agents, (e.g., an FBXO7 antibody compositions with a PD-1 inhibitory agent) described herein, is also envisioned herein. The amount of inhibitor or antibody composition in a given dosage may vary depending on the size of the individual being treated and the nature of the disorder being treated. In exemplary treatments, about 1 mg/day, 5 mg/day, 10 mg/day, 20 mg/day, 50 mg/day, 75 mg/day, 100 mg/day, 150 mg/day, 200 mg/day, 250 mg/day, 500 mg/day, or 1000 mg/day may need to be administered. These concentrations can be administered as a single dosage form or as multiple doses. Standard dose-response studies, first modeled on animals and then in clinical trials, reveal optimal dosages for specific disease states and patient populations.

It is also envisaged in the present disclosure that the amount of the therapeutic agents (e.g., FBXO7 and PD-1 inhibitors) in a given dosage may vary depending on the size of the individual being treated and the nature of the disorder being treated. Both inhibitor compositions can be administered in doses ranging from 0.1 to 15 mg as intravenous infusions every 1 to 4 weeks for 30 to 60 minutes until disease progression or unacceptable toxicity. In various embodies, the dose can be 1, 2, 3, 4, 5, 6, 7, 8, 9, 10 mg/kg. It is also clear that dosing can be modified when conventional therapeutic agents are administered in combination with the therapeutic agents of the present disclosure.

In some methods, two or more mAbs (for example, FBXO7 antibody and PD-1 antibody) with different binding specificities can be administered simultaneously, in which case the dosage of each Ab administered falls within the indicated range. Antibodies are usually administered in multiple times. The interval between single administrations can be, for example, weekly, every two weeks, every three weeks, every month, every three months or every year. The interval can also be irregular as indicated by measuring the blood level of the pharmaceutical entity (for example, FBXO7 antibody or FBXO7 siRNA) in the patient. In some methods, the dosage is adjusted to achieve a plasma Ab concentration of about 1 to 1,000 μg/mL, and in some methods a plasma Ab concentration of about 25 to 300 μg/mL. In some methods, the dosage is adjusted to achieve a plasma siRNA concentration of about 1 to 1,000 μg/mL, and in some methods a plasma siRNA concentration of about 25 to 300 g/mL.

On the other hand, the composition comprising the therapeutic agent (e.g., the FBXO7 Antibody) can be administered as a sustained release formulation, in which case less frequent administration is required. Dosage and frequency vary depending on the half-life of Ab in the patient. In general, human Abs exhibit the longest half-life, followed by humanized Abs, chimeric Abs and non-human Abs. The dosage and frequency of administration can vary depending on whether the treatment is prophylactic or therapeutic. In prophylactic applications, relatively low dosages are typically administered at relatively sparse intervals over an extended period of time. Some patients continue to receive treatment for the rest of his life. In therapeutic applications, it is often required to administer relatively high doses at relatively short intervals until the progression of the disease is reduced or terminated, preferably until the patient shows partial or complete recovery of disease symptoms. Thereafter, the patient can be administered a prophylactic regimen.

The therapeutic composition can also be delivered to the patient at multiple sites. Multiple doses can be provided simultaneously or can be administered over a period of time. In certain cases, it is beneficial to provide a continuous stream of therapeutic composition. Periodically, for example, once an hour, once a day, once every two days, twice a week, three times a week, once a week, every other week, once every three weeks, once a month, or Additional therapy can be given at longer intervals.

The actual dosage level of the active ingredient in the pharmaceutical composition of the present disclosure can be varied to obtain an amount of active ingredient effective to achieve the desired therapeutic response to the particular patient, composition and mode of administration, while not being excessively toxic to the patient. The selected dosage level depends on the activity of the particular composition of the disclosure employed, the route of administration, the time of administration, the rate of excretion of the particular compound employed, the duration of treatment, and other drugs, compounds and/or substances used in combination with the particular composition employed. The age, sex, weight, condition, general health and previous medical history of the patient being treated, and other factors. The composition of the present disclosure can be administered through one or more routes of administration using one or more of various methods. The route and/or mode of administration may vary depending on the desired outcome.

The methods herein are expected to reduce tumor size or tumor loading in a subject and/or reduce metastasis in a subject. In various embodiments, the method reduces tumor size by 10%, 20%, 30% or more. In various embodiments, the methods herein reduce tumor size by 10%, 15%, 20%, 25%, 30%, 35%, 40%, 45%, 50%, 55%, 60%, 65%, 70%, 75%, 80%, 85%, 90%, 95%, or 100% reduction. The methods herein are also expected to reduce tumor burden and reduce or prevent tumor recurrence once the cancer is in remission.

### Kits

Disclosed herein, in some embodiments, are kits for using the compositions described herein. In some embodiments, the kits disclosed herein can be used to treat a disease or condition in a subject. In some embodiments, the kits comprise an assemblage of materials or components apart from the composition.

In some embodiments, the kits described herein comprise a pharmaceutical formulation disclosed herein, comprising (i) an F-box only protein 7 (FBXO7) inhibitor, an Eyes absent homology 2 (EYA2) inhibitor, or both, (ii) a chemical entity that inhibits the interactions between FBXO7 and EYA2, or (iii) a chemical entity that interferes with the stabilization of EYA2 by FBXO7. In some embodiments, the kit described herein may further comprise an immune checkpoint molecule, such as anti-PD-1. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such as cancer. In some embodiments, the cancer comprises cancer of the lung tissue. In some embodiments, the cancer is lung cancer.

In some embodiments, the kit comprises the components for assaying the number of units of a biomolecule (e.g., FBXO7, EYA2, GAS6) synthesized, and/or released or expressed in the body of a subject. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use can be included in the kit. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

Optionally, the kit also contains other useful components such as diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s).

### EXAMPLES

### Example 1: FBXO7 Maintains Cancer Cell Mesenchymal and Immune Evasion Phenotypes

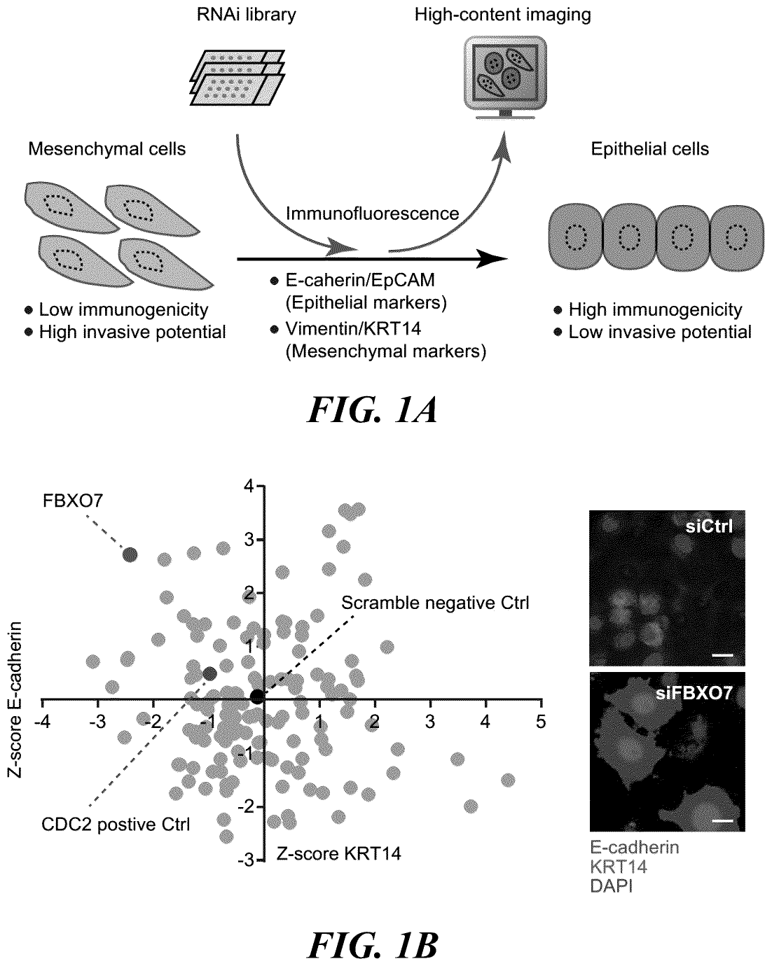

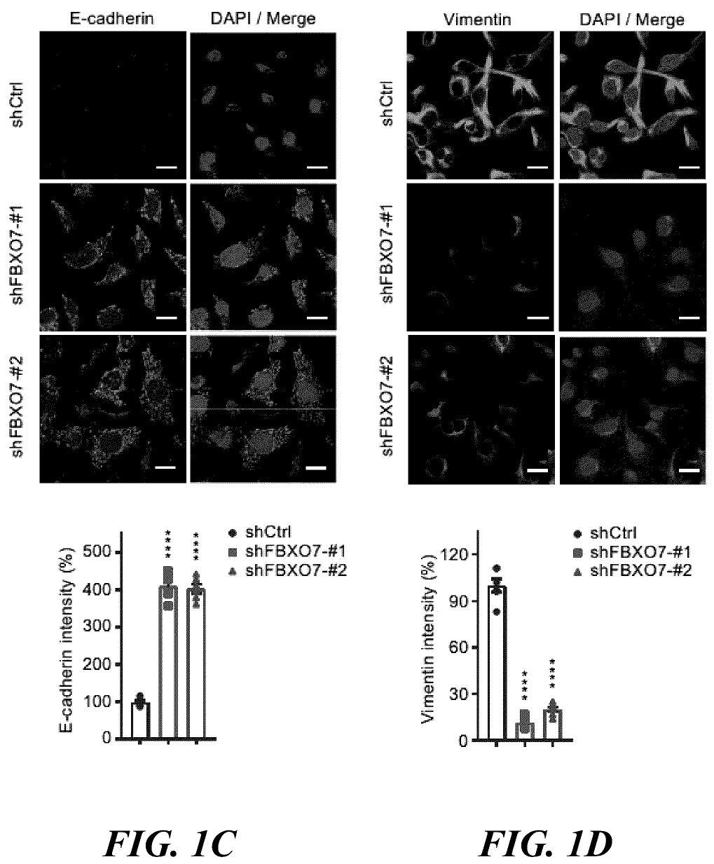

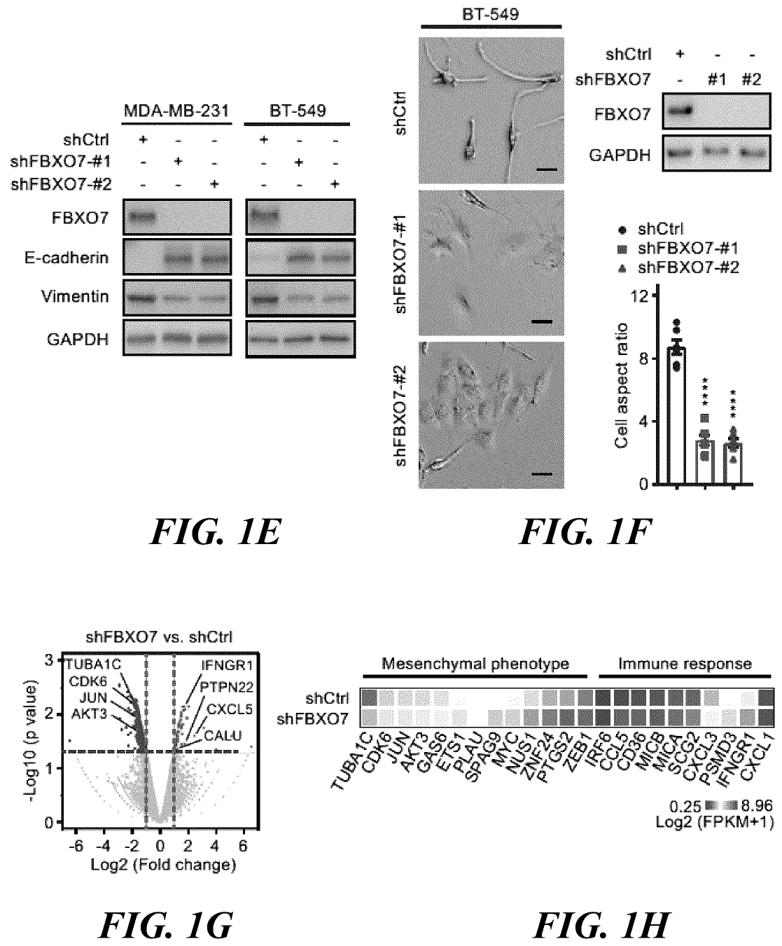

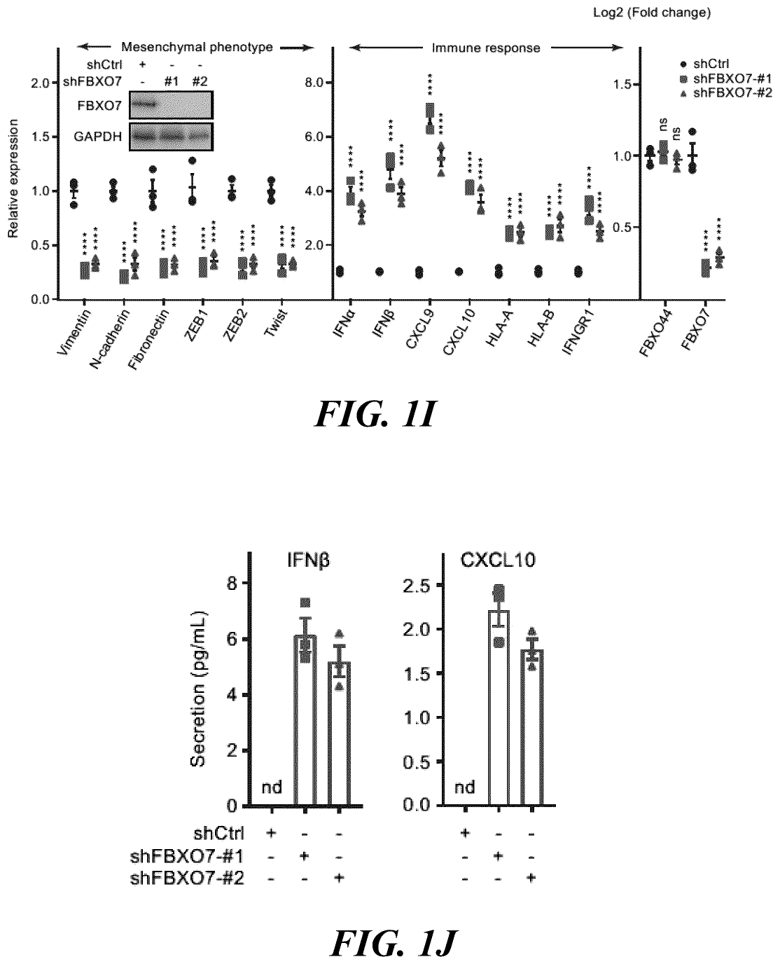

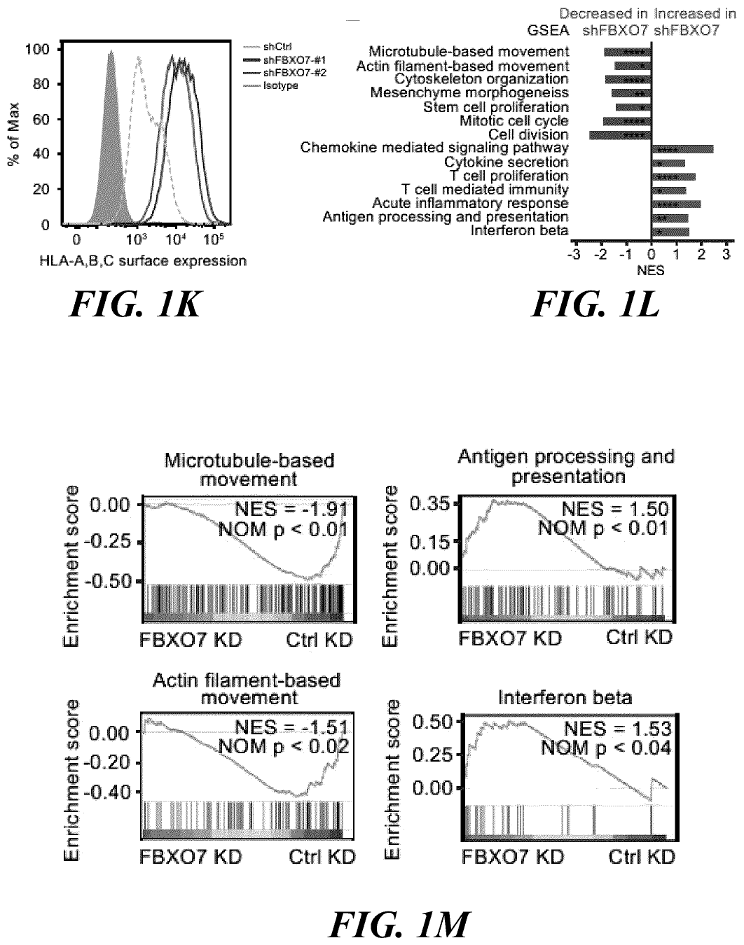

To identify mesenchymal maintenance regulators in cancer cells, an RNA interference (RNAi) screen was performed for genes whose knockdown (KD) decreased mesenchymal markers and increased epithelial markers in TNBC cell lines that displayed enhanced mesenchymal and CSC characteristics (FIG. 1A). The screen focused on ubiquitin ligases since their role in regulating mesenchymal phenotypes was largely unexplored. FBXO7 was identified as a top hit (FIG. 1B). FBXO7 is a member of the F-box protein family, the members of which function as substrate recognition factors for the SKP1-Cullin-F-box (SCF) E3 ubiquitin ligase. Immunofluorescence (IF) and immunoblotting confirmed FBXO7 KD in mesenchymal cancer cells reduced mesenchymal and increased epithelial markers (FIG. 1C-1E). FBXO7 KD induced a flat, cuboidal morphology, consistent with cells undergoing a mesenchymal-to-epithelial (MET) transition (FIG. 1F).

RNA-sequencing (RNA-seq) revealed that FBXO7 KD decreased the expression of EMT-associated genes (e.g., JUN, MYC, ZEB1, TUBA1C, CDK6, AKT3, and GAS6) and increased immune-stimulatory genes (e.g., IFNGR1, CXCL5, and MICA/MICB (MHC class I chains)) (FIGS. 1G and 1H). Quantitative RT-PCR (RT-qPCR) analysis confirmed FBXO7 KD downregulated various mesenchymal markers, including EMT transcription factors ZEB1, ZEB2, and TWIST, and upregulated immune-stimulatory IFNα and IFNβ, chemokines CXCL9 and CXCL10, and HLA-A and HLA-B (FIG. 1I). ELISA and flow cytometry verified increased IFNβ and CXCL10 secretion and enhanced HLA-A/B/C surface expression on FBXO7 KD cells, respectively (FIGS. 1J and 1K). Gene set enrichment analysis (GSEA) of FBXO7 KD RNA-seq showed pathways associated with cell movement, stem cell maintenance, and cell division were downregulated, while immune-stimulatory pathways, including IFNβ, antigen processing and presentation, cytokine secretion, and acute inflammatory response, were upregulated (FIGS. 1L, 1M, S1B, and S1C). Collectively, these data identify FBXO7 as a mesenchymal maintenance regulator in cancer cells and demonstrate its targeting induces immune-stimulatory phenotypes that may promote susceptibility to adaptive immunity.

### Example 2: FBXO7 Binds and Stabilizes SIX1 Co-Transcriptional Regulator EYA2

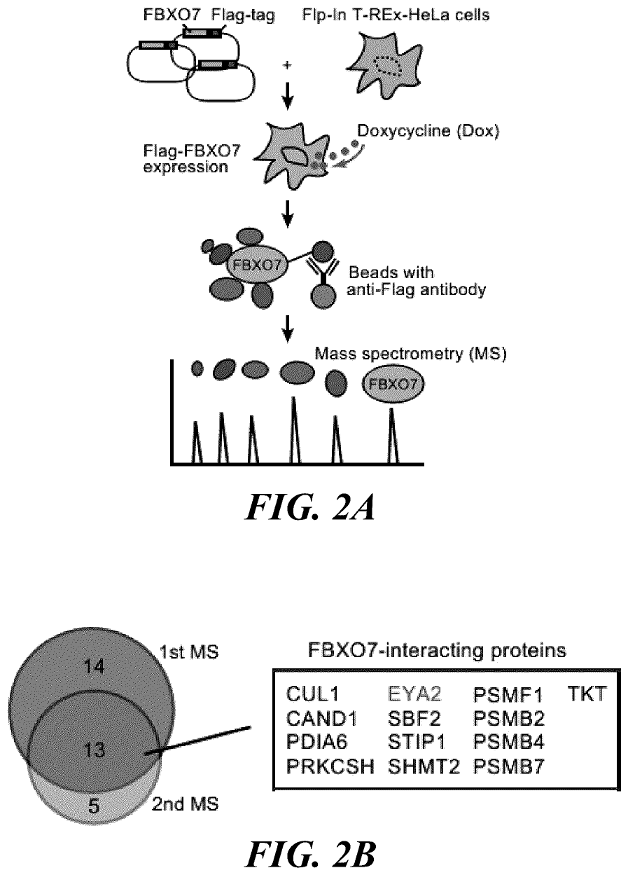

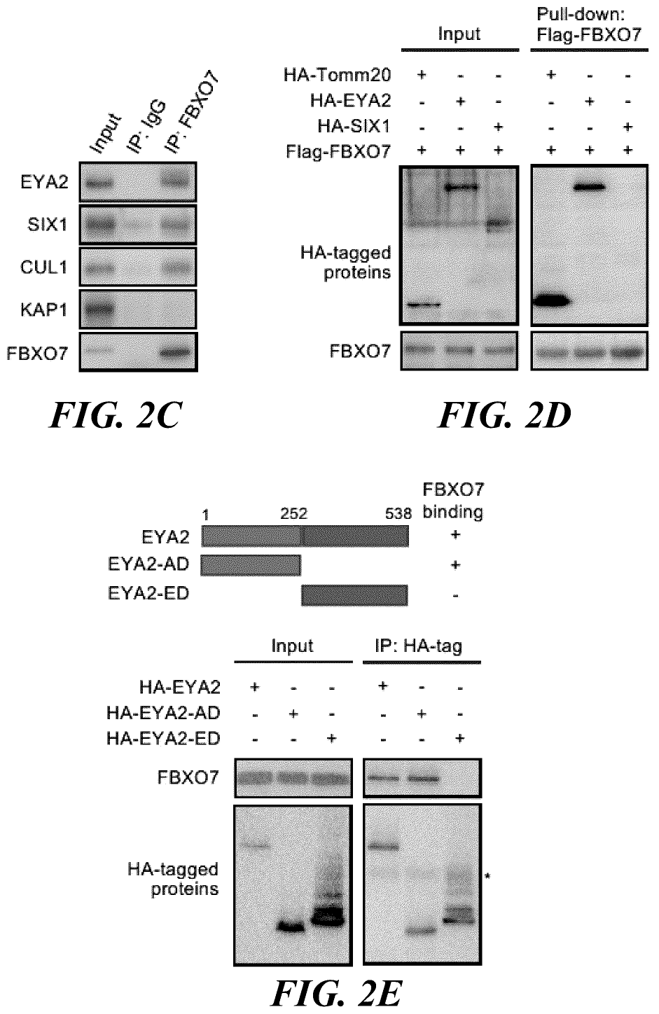

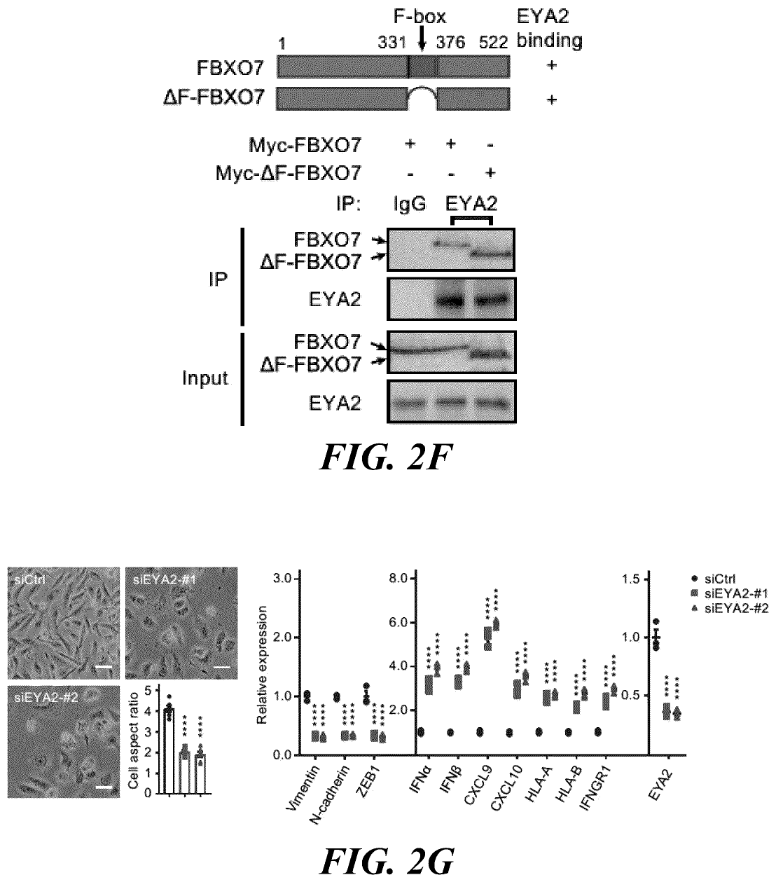

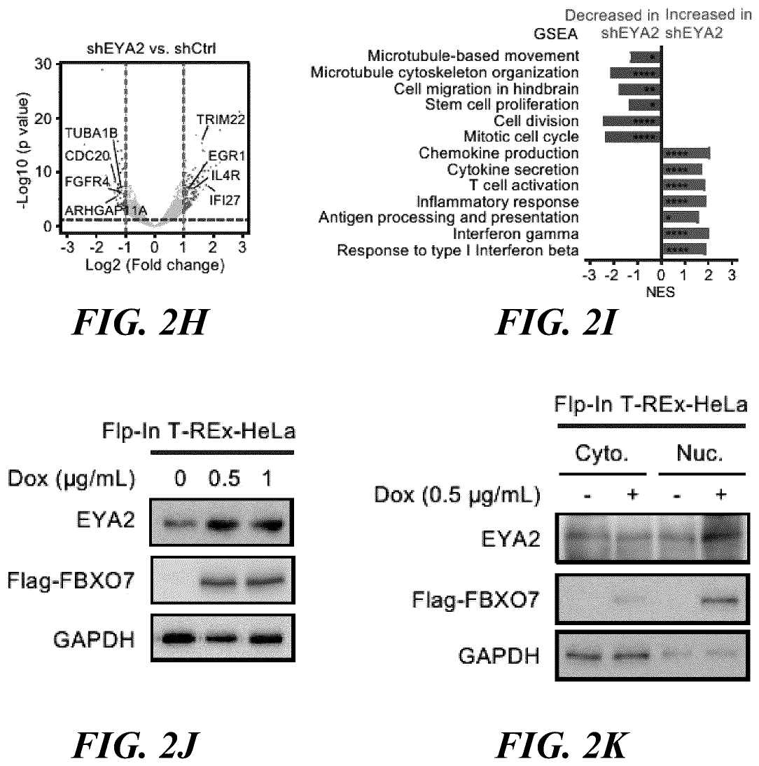

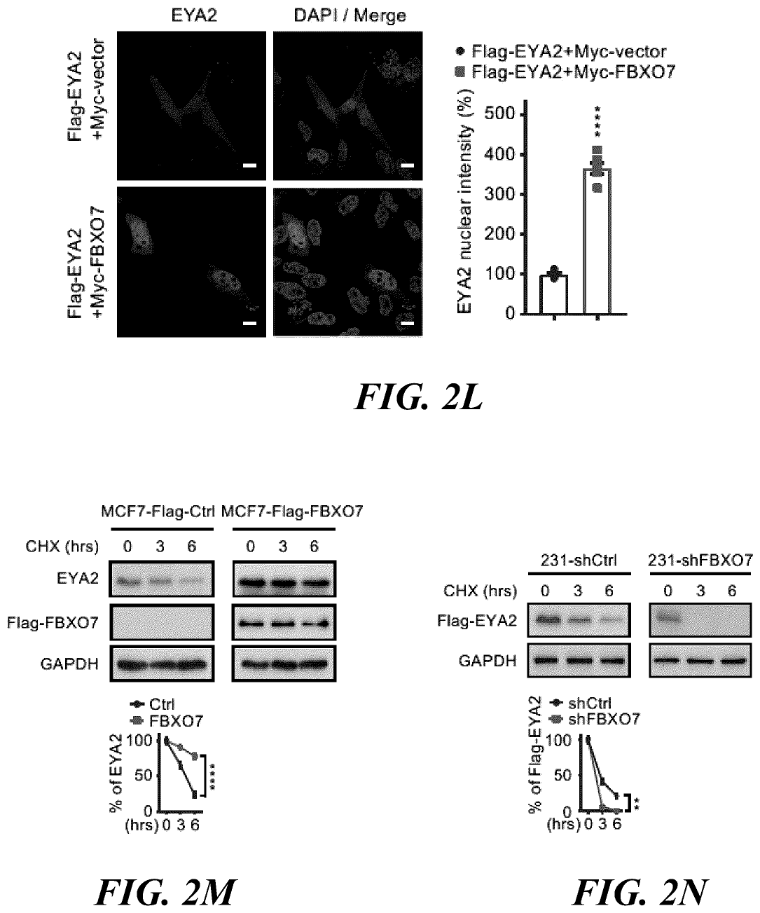

To gain insight into how FBXO7 maintains mesenchymal phenotypes and immune evasion of cancer cells, protein mass spectrometry was conducted to identify putative interacting proteins (FIG. 2A). Eyes Absent Homolog 2 (EYA2), a co-activator of the SIX family of homeobox transcription factors with Tyr phosphatase activity, was one of the most abundant FBXO7-interacting proteins (FIG. 2B). Immunoprecipitation (IP) and in vitro pull-down assays confirmed that FBXO7 interacted with EYA2, but not directly with co-transcriptional partner SIX1 (FIGS. 2C and 2D). EYA2's transactivation domain (AD, aa1-252) but not EYA domain (ED, aa253-538), which interacts with SIX proteins, was required for FBXO7 interaction (FIG. 2E). FBXO7's F-box motif was dispensable for EYA2 interaction (FIG. 2F). Further, treatment of cells with NEDDylation inhibitor MLN4924 did not disrupt FBXO7-EYA2 binding (data not shown), indicating SCF activity was not required.

EYA2 KD induced a flat, cuboidal morphology, decreased mesenchymal gene expression, and induced immune-stimulatory genes (FIGS. 2G-2I), phenocopying FBXO7 KD. Similar effects were observed for SIX1 KD cells (data not shown). Treatment with EYA2 Tyr phosphatase inhibitor, MLS000544460 (hereafter, “EYA2i”), induced similar effects on gene expression and cell morphology (data not shown). Expression of WT EYA2, but not Tyr phosphatase mutant EYA2D274N or SIX binding mutant EYA2A532R, largely rescued the decreased mesenchymal and increased immune-stimulatory genes in FBXO7 KD cells (data not shown).

FBXO7 expression increased EYA2 steady-state levels (FIG. 2J). Cell fractionation and IF revealed FBXO7 predominantly localized to the nucleus and FBXO7 expression increased nuclear EYA2 (FIGS. 2K and 2L). FBXO7 expression stabilized EYA2 in epithelial MCF7 breast cancer cells with cycloheximide (CHX) treatment (FIG. 2M). Conversely, FBXO7 KD destabilized EYA2 in mesenchymal MDA-MB-231 breast cancer cells (FIG. 2N). FBXO7 KD had no effect on EYA2 transcription (Figure S2E). Together, these data indicate that FBXO7 binds, stabilizes, and promotes nuclear accumulation of EYA2 in cancer cells.

### Example 3: FBXO7 Protects EYA2 from SCFFBXW7-Dependent Degradation

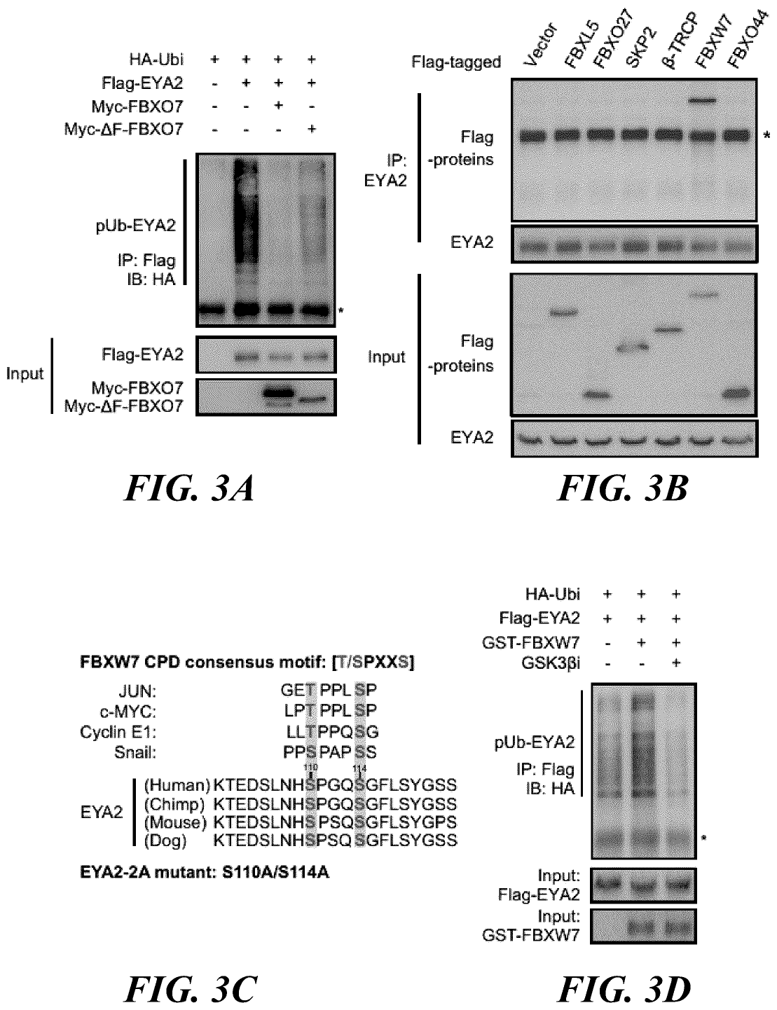

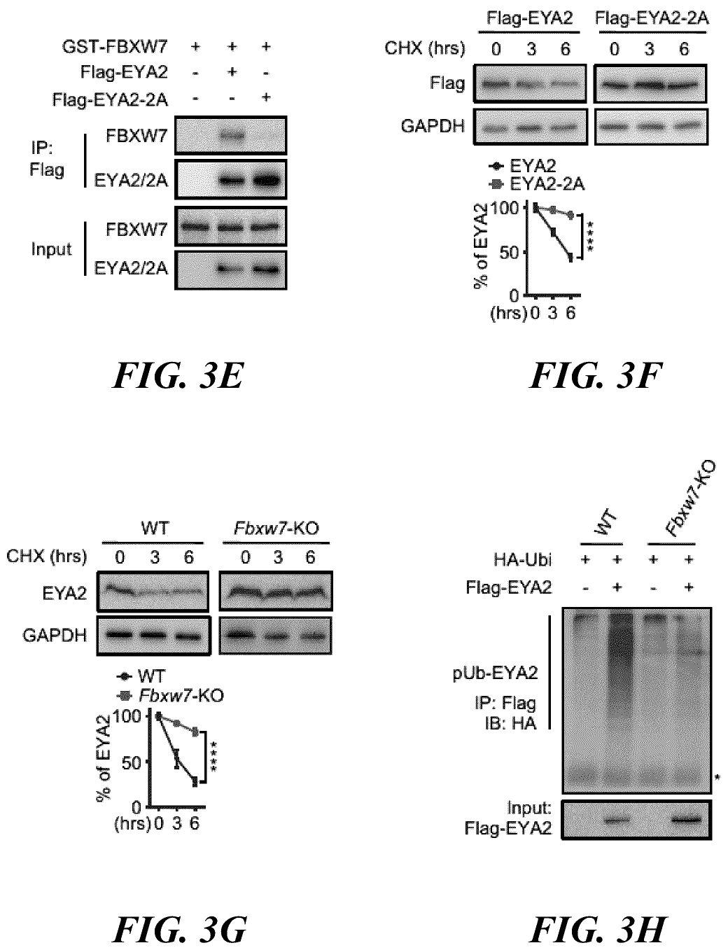

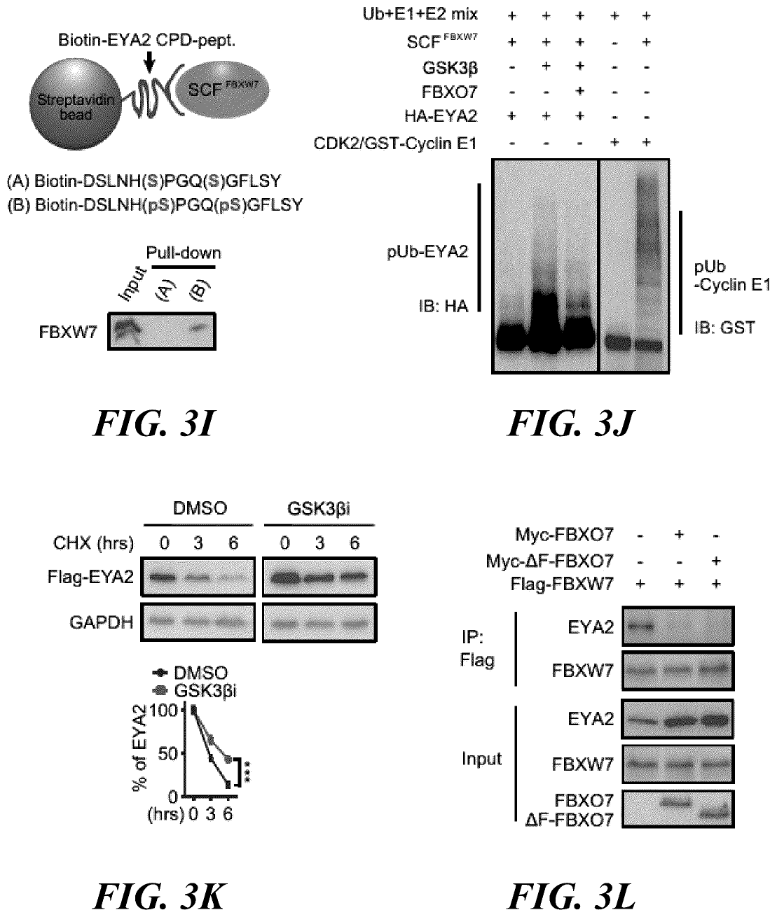

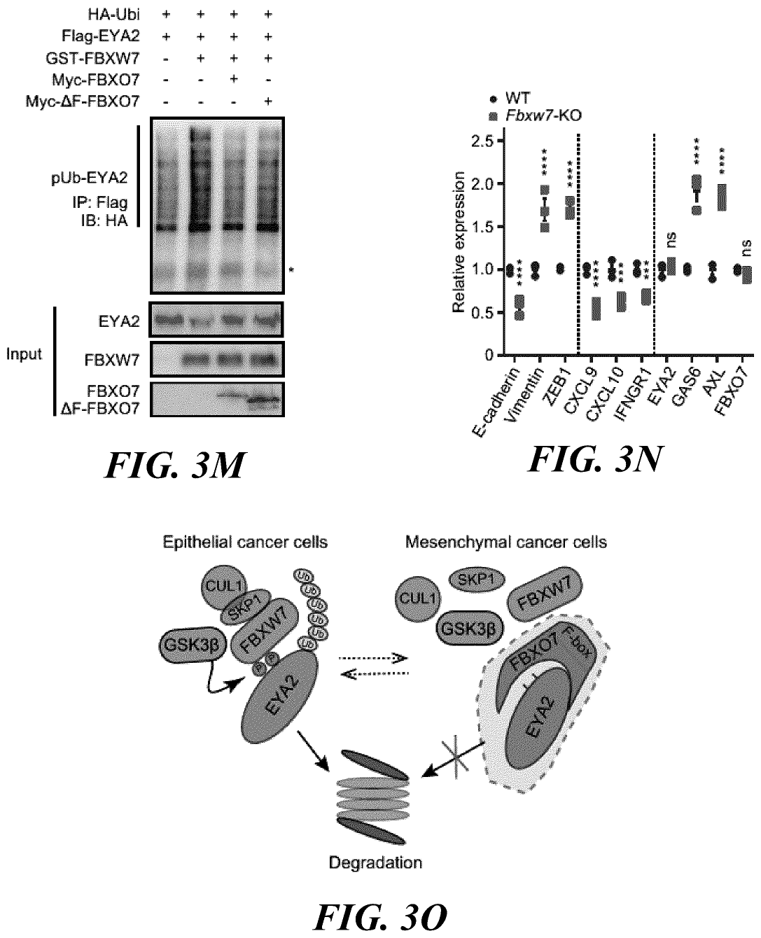

EYA2 increased with 26S proteasome inhibitor treatment, indicating regulation by ubiquitin-dependent proteolysis (data not shown). In vivo ubiquitylation reactions showed EYA2 was efficiently polyubiquitylated in cells, which was attenuated by FBXO7 expression (FIG. 3A). Expression of F-box-deleted FBXO7 (ΔF-FBXO7), that did not bind SCF component CUL1 (data not shown), increased EYA2 stability and decreased polyubiquitylation (FIGS. 3A and S3C), suggesting FBXO7 might interfere with EYA2 polyubiquitylation via an SCF-independent mechanism. In support, FBXO7 or ΔF-FBXO7 expression rescued the decreased mesenchymal and increased immune-stimulatory phenotypes in FBXO7 KD cells (data not shown). Additionally, in vitro ubiquitylation reactions failed to show EYA2 was a substrate of SCFFBXO7 ubiquitylation activity (data not shown).

MLN4924 treatment increased EYA2 levels, indicating regulation by a Cullin-RING E3 ubiquitin ligase (data not shown). IP showed that EYA2 selectively interacted with FBXW7 in a panel of randomly selected F-box proteins (FIG. 3B). Analysis of EYA2 revealed that it harbored a conserved CDC4 phosphodegron (CPD) (FIG. 3C), the substrate recognition motif for the SCFFBXW7 ubiquitin ligase. FBXW7 expression enhanced EYA2 K48-linked but not K63-linked polyubiquitylation (FIG. 3D), indicating SCFFBXW7 regulated EYA2 degradation. IP confirmed EYA2's CPD was required for FBXW7 binding (FIG. 3E). In contrast, FBXO7-EYA2 binding was independent of EYA2's CPD (data not shown). Further, FBXO7 did not bind SCFFBXW7 substrates MYC, MCL-1, or Cyclin E1 or influence their steady-state levels (data not shown). CHX experiments showed that CPD mutant EYA2 (designated EYA2-2A) was stabilized compared to WT EYA2 (FIG. 3F). EYA2-2A also displayed decreased polyubiquitylation in vivo (data not shown). In isogenic WT and Fbxw7 knockout (KO) cell lines, the steady-state level and stability of EYA2 increased and polyubiquitylation decreased in the absence of Fbxw7 (FIGS. 3G, 3H).

SCFFBXW7 substrate recognition is generally triggered by GSK3β-mediated phosphorylation of the substrate CPD. Recombinant SCFFBXW7 bound a synthetic peptide corresponding to phosphorylated EYA2 CPD (FIG. 3I). Further, in vitro kinase assays showed GSK3β phosphorylated the EYA2 CPD (data not shown). To determine if EYA2 degradation was regulated by GSK3β, we first confirmed endogenous GSK3 β interacted with EYA2 in vivo by IP (data not shown). Treatment with GSK3β inhibitor (GSK3βi) disrupted FBXW7-EYA2 binding (data not shown), but not FBXO7-EYA2 binding (data not shown). GSK3β stimulated SCFFBXW7-mediated polyubiquitylation of EYA2 in in vitro ubiquitylation reactions (FIG. 3J). Consistently, GSK3βi stabilized EYA2 and inhibited its polyubiquitylation in vivo (FIGS. 3D, 3K). Therefore, EYA2 is degraded by SCFFBXW7 in a GSK3β-dependent manner.

To understand how FBXO7 protects EYA2 from SCFFBXW7-mediated degradation, it was examined whether FBXO7 influenced FBXW7's recognition of EYA2. IP showed FBXO7 or ΔF-FBXO7 expression diminished FBXW7-EYA2 binding (FIG. 3L). Conversely, FBXO7 KD enhanced FBXW7-EYA2 binding (data not shown). Expression of FBXO7 or ΔF-FBXO7 decreased EYA2 polyubiquitylation by SCFFBXW7 (FIG. 3M). Corroborating these data, Fbxw7 KO cells expressed increased mesenchymal and decreased immune-stimulatory genes compared to WT counterparts (FIG. 3N). Further, FBXW7 KD largely rescued the decreased mesenchymal and increased immune-stimulatory genes expressed in FBXO7 KD cells (data not shown). Collectively, these data indicate that FBXO7 blocks FBXW7 recognition of EYA2, thus antagonizing GSK3β-SCFFBXW7-mediated degradation (FIG. 3O).

### Example 4: FBXO7 Promotes EYA2/SIX1-Mediated Induction of AXL Ligand GAS6

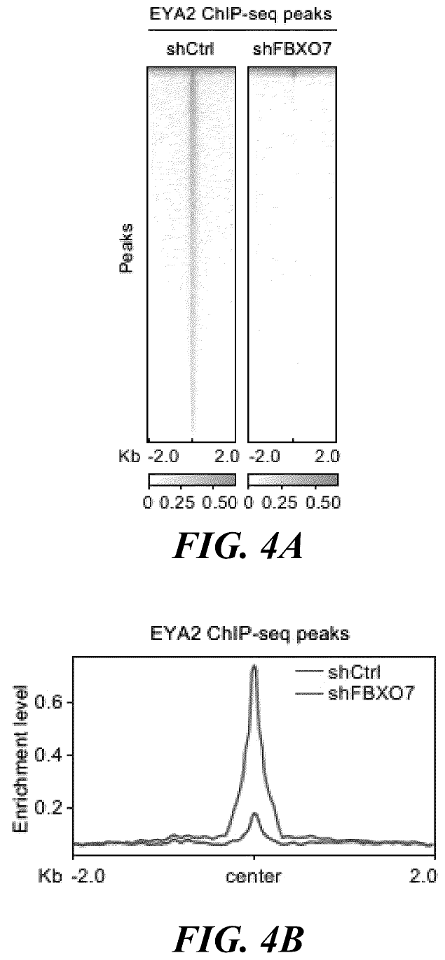

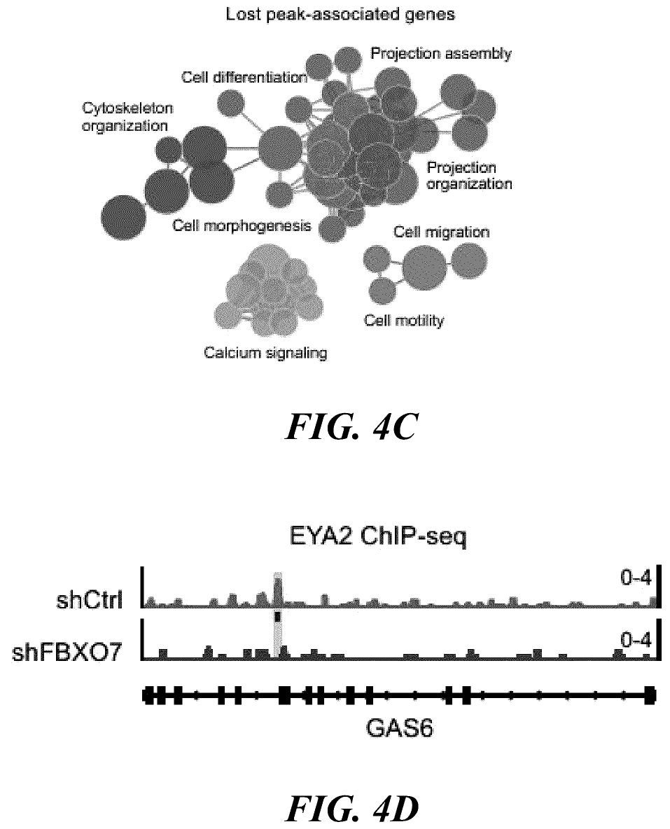

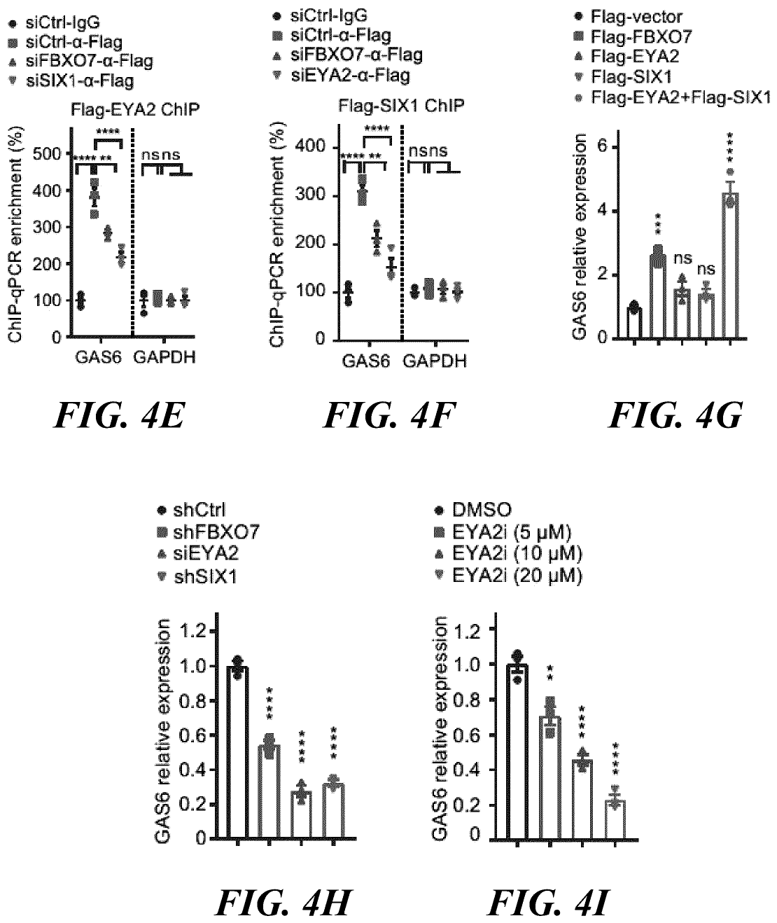

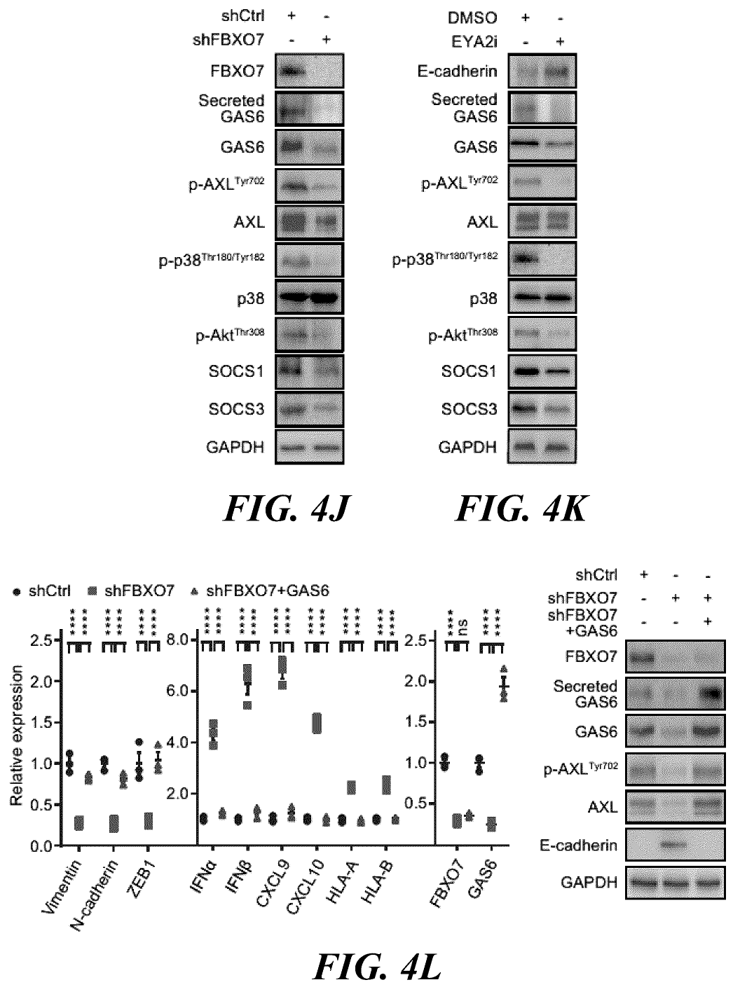

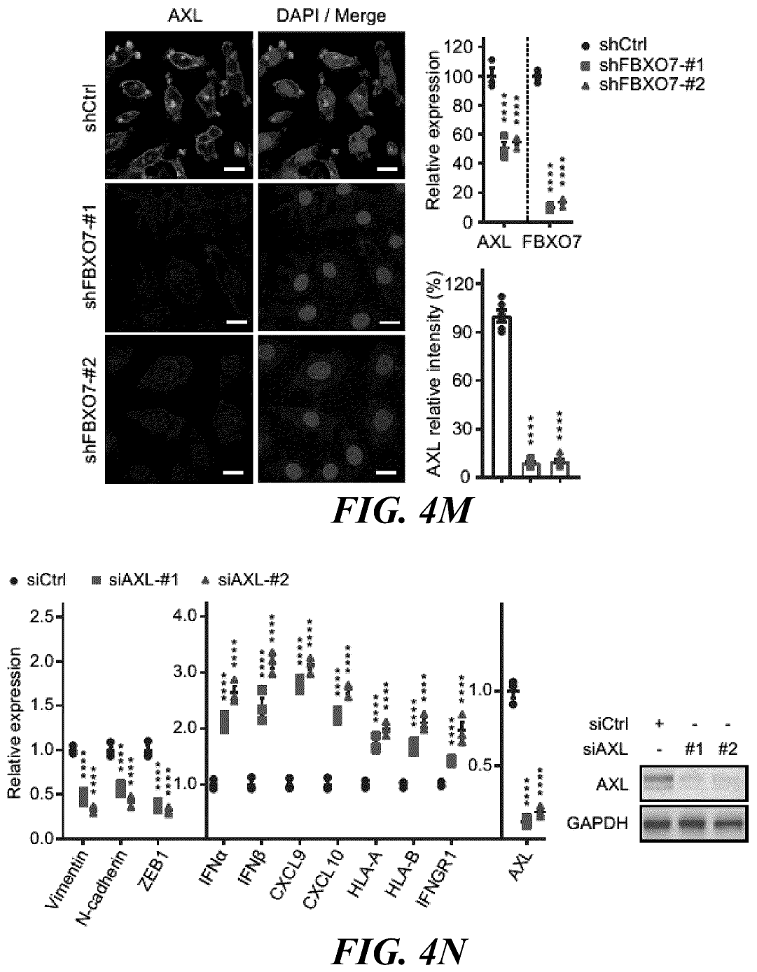

To determine how the FBXO7/EYA2-SCFFBXW7 axis maintains mesenchymal and immune evasion phenotypes of cancer cells, chromatin IP sequencing (ChIP-seq) profiles of EYA2 binding in control and FBXO7 KD cells (FIGS. 4A and 4B) were compared. FBXO7 KD decreased EYA2 binding at various genomic regions, including promoters, exons, and introns (data not shown). Gene ontology (GO) analysis revealed that genes with reduced EYA2 binding upon FBXO7 KD were enriched in cell migration and motility, cytoskeleton organization, and projection assembly and organization, among others (FIG. 4C), consistent with a proposed role for FBXO7 in maintaining cancer cell mesenchymal signatures. Among the top-ranked genes that bound EYA2 at the promoter, displayed decreased EYA2 binding and expression upon FBXO7 KD, and had defined roles in EMT were ubiquitin-conjugating enzyme UBE2T, histone methyltransferase PRDM2, and AXL autocrine/paracrine GAS6 (FIG. 4D). GAS6 was chosen for further analysis because of the link between AXL and mesenchymal phenotypes in TNBC cells. ChIP confirmed that EYA2 and SIX1 bound the Gas6 promoter and binding was diminished by KD of FBXO7 or co-transcriptional counterpart (FIGS. 4E and 4F). EYA2-2A also bound the Gas6 promoter (data not shown), indicating independence of CPD phosphorylation. Further, FBXO7 bound the Gas6 promoter and FBXO7 or ΔF-FBXO7 expression stimulated EYA2 binding (data not shown). RT-qPCR analysis showed expression of EYA2+SIX1 or FBXO7 promoted GAS6 expression in epithelial MCF7 cells (FIG. 4G). Expression of EYA2-2A+SIX1 or ΔF-FBXO7 also induced GAS6 expression (data not shown). Conversely, KD of FBXO7, EYA2, or SIX1, or EYA2i treatment decreased GAS6 expression in mesenchymal MDA-MB-231 cells (FIGS. 4H and 4I). Consistently, FBXO7 KD or EYA2i treatment decreased total and secreted GAS6 protein (data not shown). GAS6 expression largely rescued the decreased mesenchymal and increased immune-stimulatory genes in FBXO7 KD cells (FIG. 4L).

In line with the role of GAS6 as an AXL ligand, FBXO7 KD decreased total AXL and phospho-AXLTyr702 and decreased AXL surface expression (FIGS. 4J and 4M), indicating inhibition of AXL signaling. FBXO7 KD also decreased phospho-p38Thr180/Tyr182, phospho-AKTT308, and SOCS1 and SOCS3, downstream effectors of AXL signaling (FIG. 4J). EYA2i treatment also suppressed AXL signaling (FIG. 4K). GAS6 expression rescued the decreased total and phospho-AXLTyr702 in FBXO7 KD cells (FIG. 4L). Consistent with a role of SCFFBXW7 in antagonizing FBXO7 functions, FBXW7 KD rescued the decreased GAS6 and attenuated AXL signaling in FBXO7 KD cells (data not shown). RT-qPCR showed that AXL KD phenocopied FBXO7/EYA2 KD in suppressing mesenchymal and promoting immune-stimulatory genes (FIG. 4N). Expression of proinflammatory cytokine suppressor and AXL signaling effector SOCS1 partially rescued the increased IFNα/β, CXCL9/10, and HLA-A/B induced by FBXO7 KD (FIG. 4O). Further, GAS6 expression rescued the increased HLA-A/B/C surface expression on FBXO7 KD cells (data not shown). Collectively, these data indicate that FBXO7 promotes mesenchymal and immune evasion phenotypes in cancer cells, at least in part, by stimulating EYA2/SIX1-GAS6 mediated activation of AXL signaling.

### Example 5: Targeting FBXO7 Suppresses Mesenchymal Phenotypes of Cancer Cells

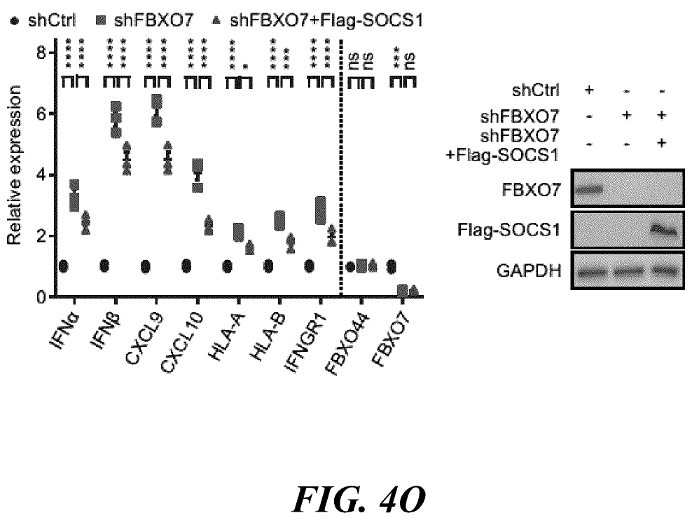

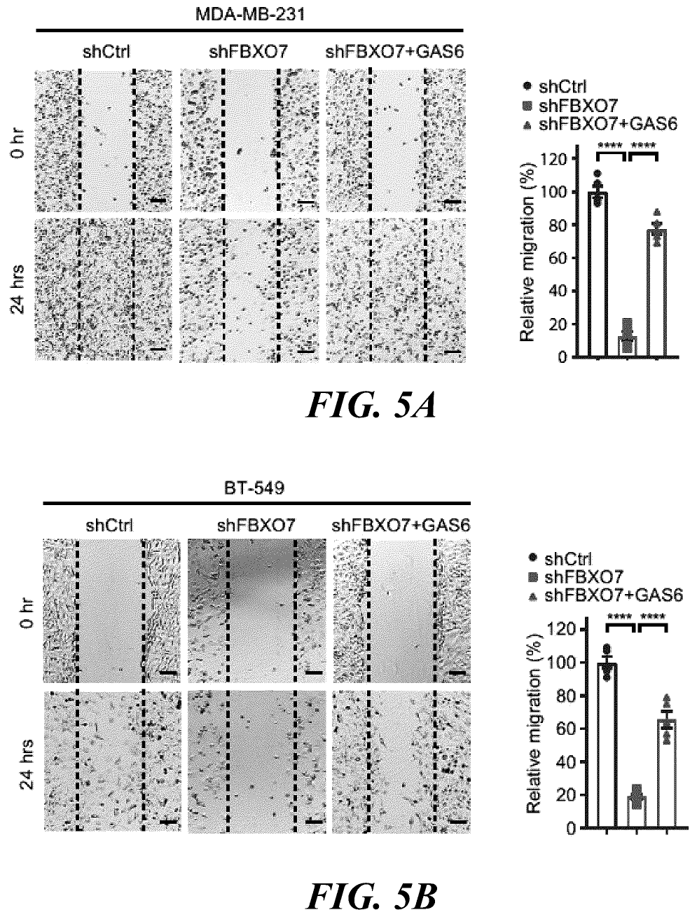

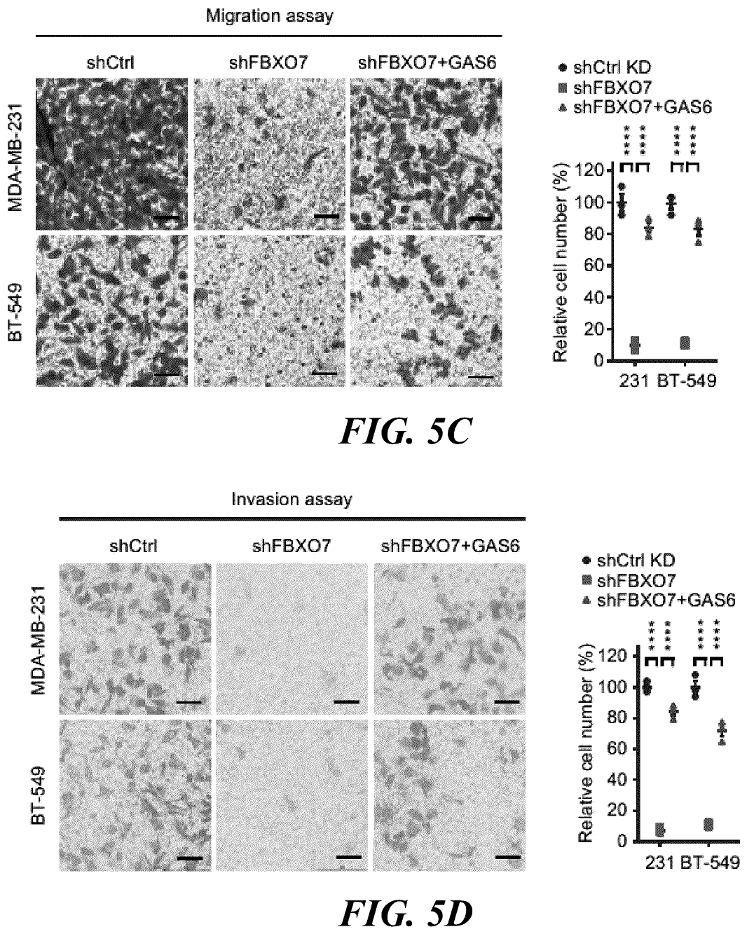

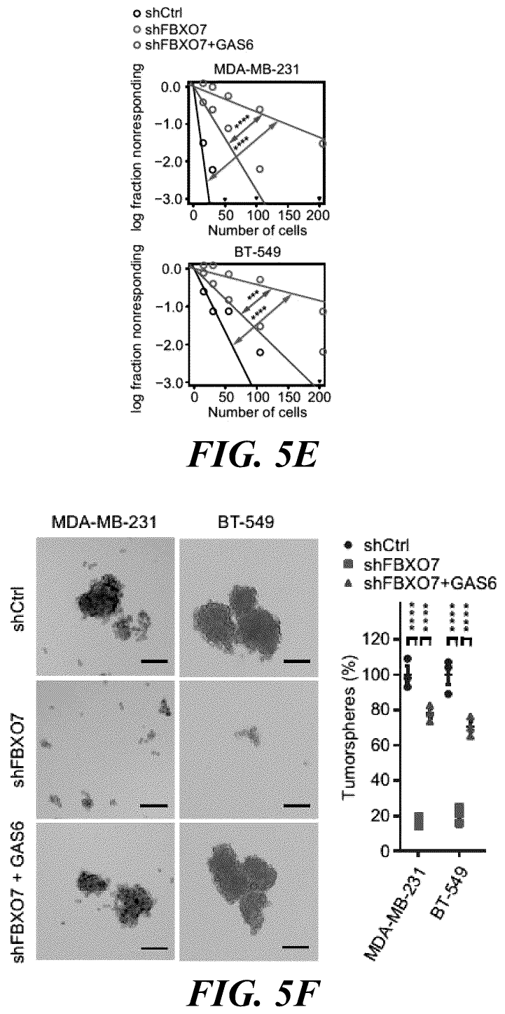

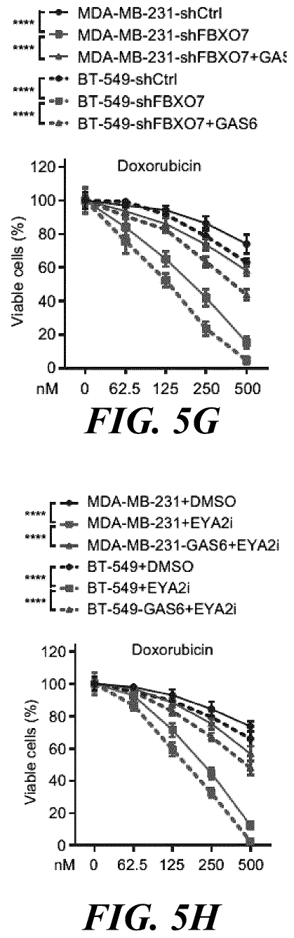

FBXO7 KD diminished mesenchymal cancer cell migration and invasion in in vitro assays (data not shown). These FBXO7 KD effects were largely rescued by GAS6 expression (FIGS. 5A-5D). FBXO7 KD also decreased the frequency and self-renewal capacity of CSCs in extreme limiting dilution assays (ELDA) (data not shown) and reduced tumor sphere formation in vitro (data not shown). Again, these phenotypes were largely rescued by GAS6 expression (FIGS. 5E and 5F). FBXO7 KD or EYA2i treatment sensitized cancer cells to doxorubicin treatment (data not shown), partially rescued by GAS6 expression (FIGS. 5G and 5H). Consistent with an antagonistic role of SCFFBXW7, FBXW7 KD partially rescued the reduced migration of FBXO7 KD cells and enhanced EYA2i sensitivity (data not shown). Together, these data show that targeting FBXO7/EYA2 suppressed mesenchymal phenotypes of cancer cells largely dependent on reduced GAS6 expression.

### Example 6: FBXO7/EYA2 Inhibition Attenuates Tumor Growth, Boosts Antitumor Immunity, and Enhances ICB Therapy Response

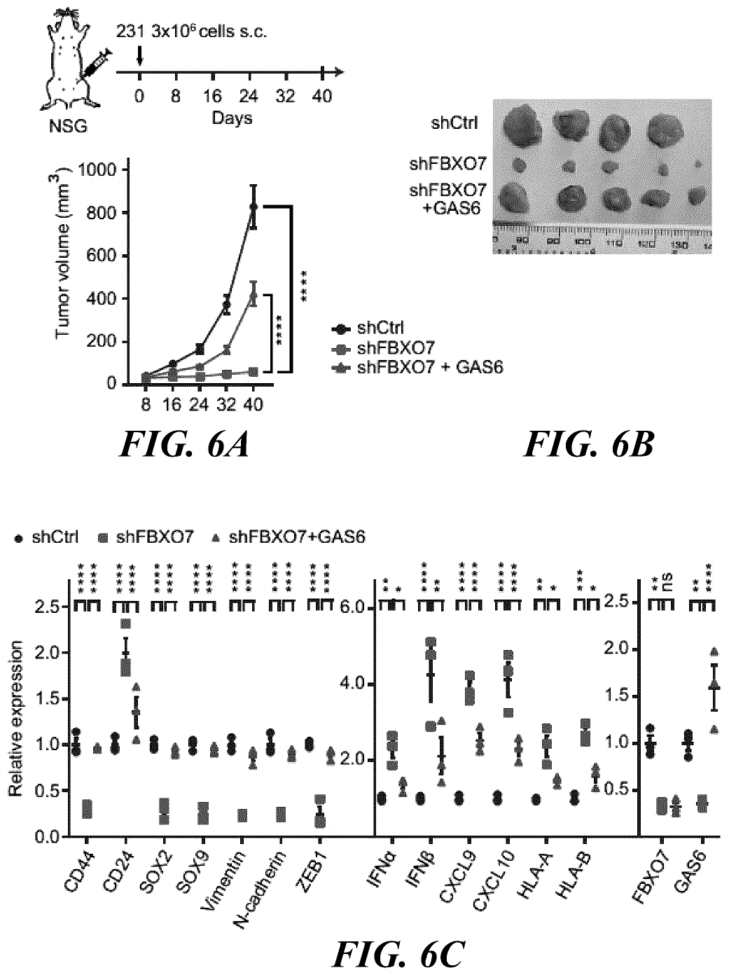

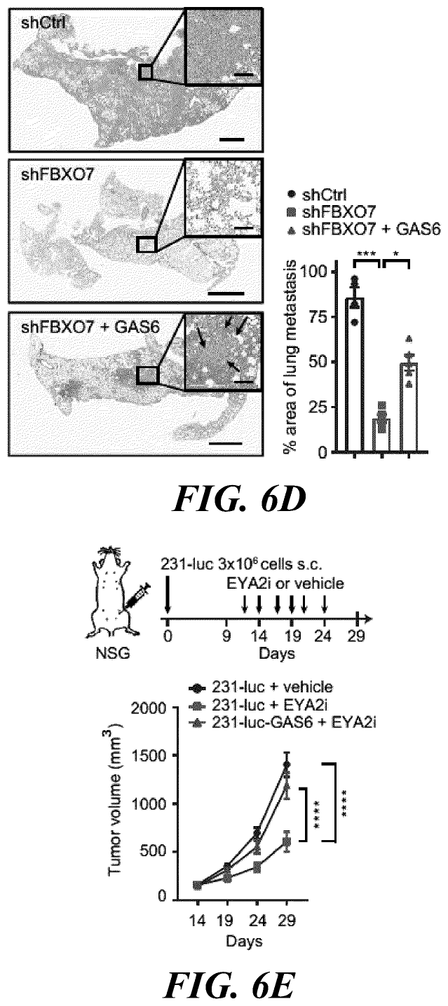

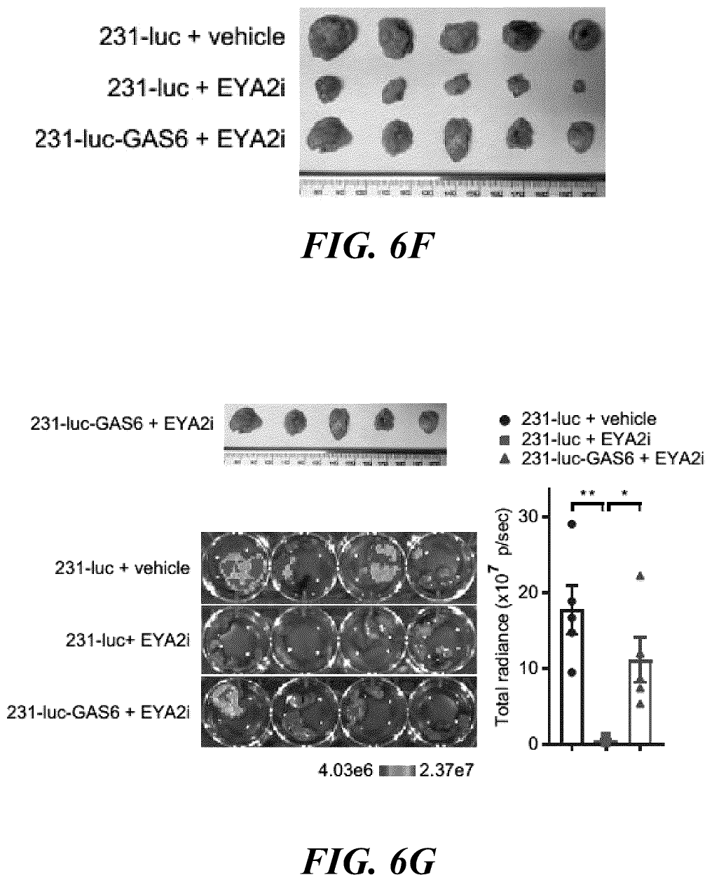

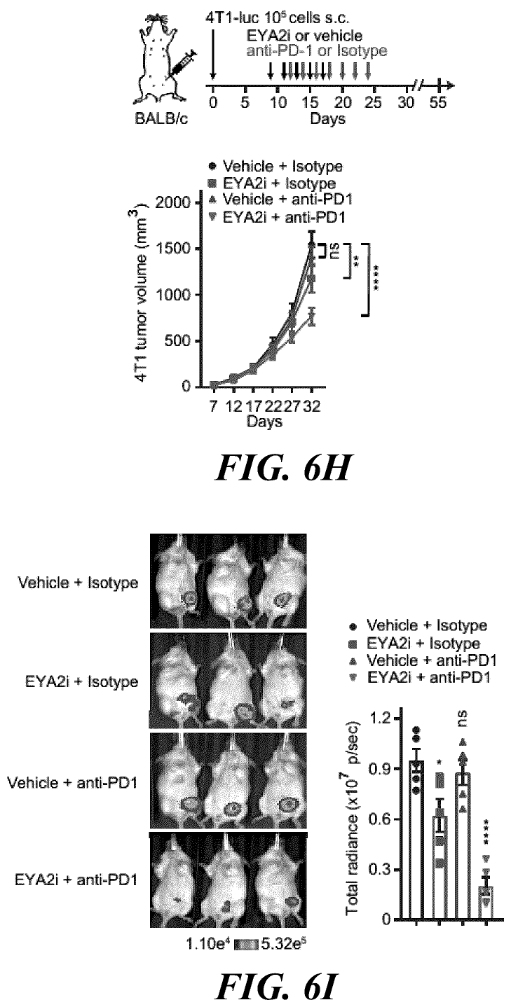

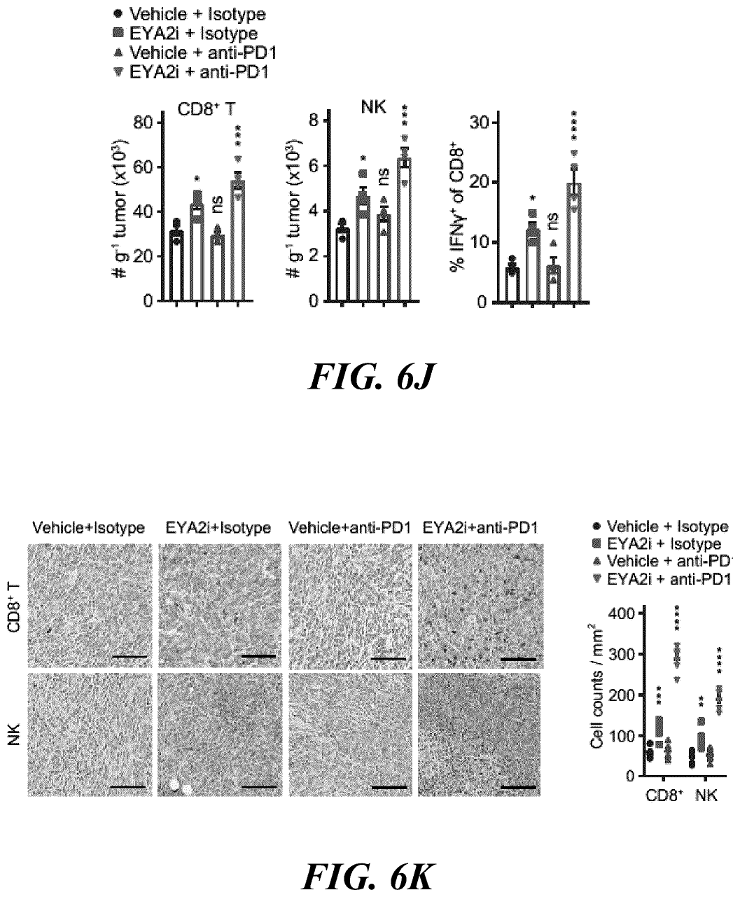

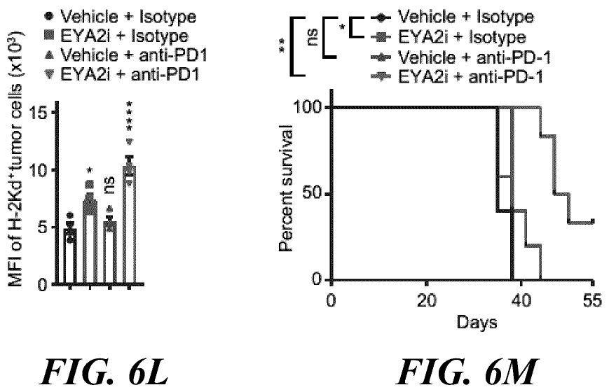

FBXO7 KD restricted MDA-MB-231 orthotopic breast tumor growth in immunocompromised mice, which was partially rescued by GAS6 expression (FIGS. 6A and 6B). FBXO7 KD tumors displayed decreased mesenchymal and increased immune-stimulatory gene expression and reduced CSC markers, which were partially rescued by GAS6 expression (FIG. 6C). Mice bearing FBXO7 KD breast tumors displayed reduced lung metastases (FIG. 6D). FBXO7 KD decreased the metastatic seeding capability of MDA-MB-231-luc cells in immunodeficient mice (data not shown).

To assess if the FBXO7/EYA2-SCFFBXW7 axis could be pharmacologically targeted for mesenchymal tumor treatment, immunocompromised mice bearing orthotopic MDA-MB-231-luc breast tumors were treated with EYA2i revealing diminished tumor cell autonomous growth and lung metastases, which were partially rescued by GAS6 expression (FIGS. 6E-6G). These data, and those described above, demonstrate stand-alone antitumor activity of targeting FBXO7/EYA2, consistent with EYA2 Tyr phosphatase activity being crucial for cancer cell migration, invasion, and metastasis.

Given that FBXO7/EYA2 inhibition enhanced cancer cell immunogenicity in vitro, we next examined if EYA2i treatment could overcome resistance to ICB therapy. Immunocompetent mice bearing 4T1-luc TNBCs were treated with vehicle or EYA2i and isotype IgG (control) or anti-PD-1 antibody (FIG. 6H). 4T1-luc tumors exhibited poor response to anti-PD-1 treatment (FIGS. 6H and 6I), corroborating a previous report (Bertrand et al., 2017). EYA2i treatment modestly reduced 4T1-luc tumor growth. In contrast, EYA2i+anti-PD-1 antibody promoted a more robust antitumor response. Flow cytometric and immunohistochemical (IHC) analyses showed that EYA2i stimulated the intertumoral infiltration of CD8+T and NK cells, which was further enhanced by anti-PD-1 antibody (FIGS. 6J, 6K and S6C). EYA2i+anti-PD-1 antibody also increased IFNγ+CD8+ T cells, indicating a functional immune response (FIG. 6J). EYA2i treatment increased H-2Kd MHC class I alloantigen surface expression on 4T1-luc tumor cells, which was further enhanced by anti-PD-1 antibody (FIG. 6L). EYA2i+anti-PD-1 treatment increased mouse survival (FIG. 6M). Therefore, targeting FBXO7/EYA2 suppresses mesenchymal phenotypes and synergizes with ICB therapy in cancer treatment.

### Example 7: FBXO7 Associates with Mesenchymal and Immune-Suppressive Phenotypes in Cancer Patient Datasets

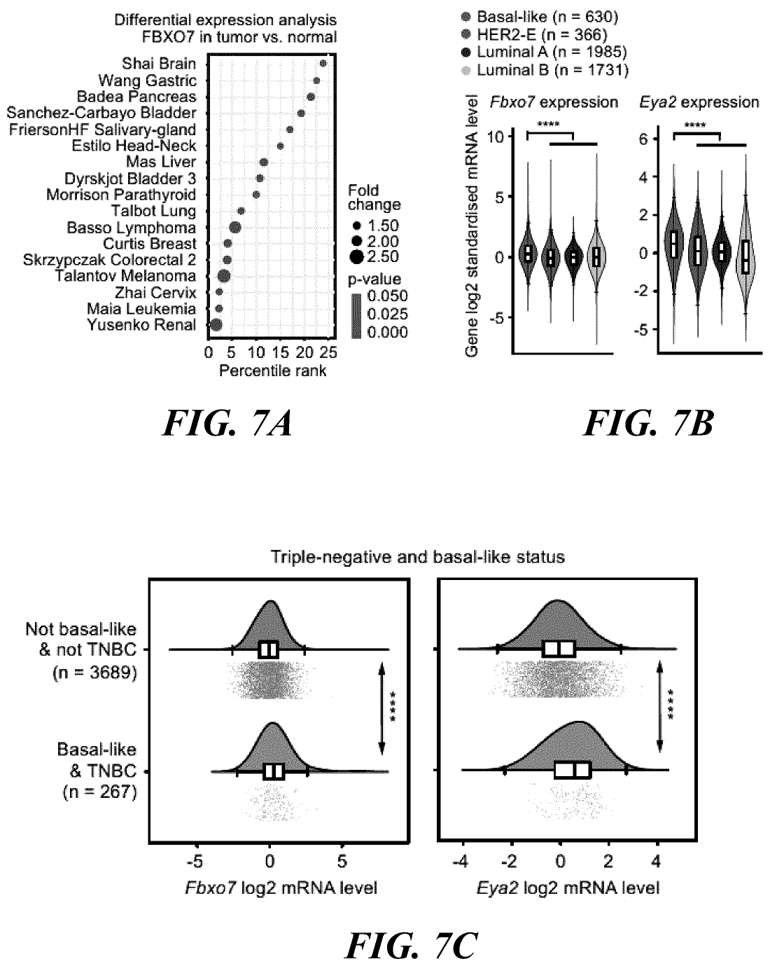

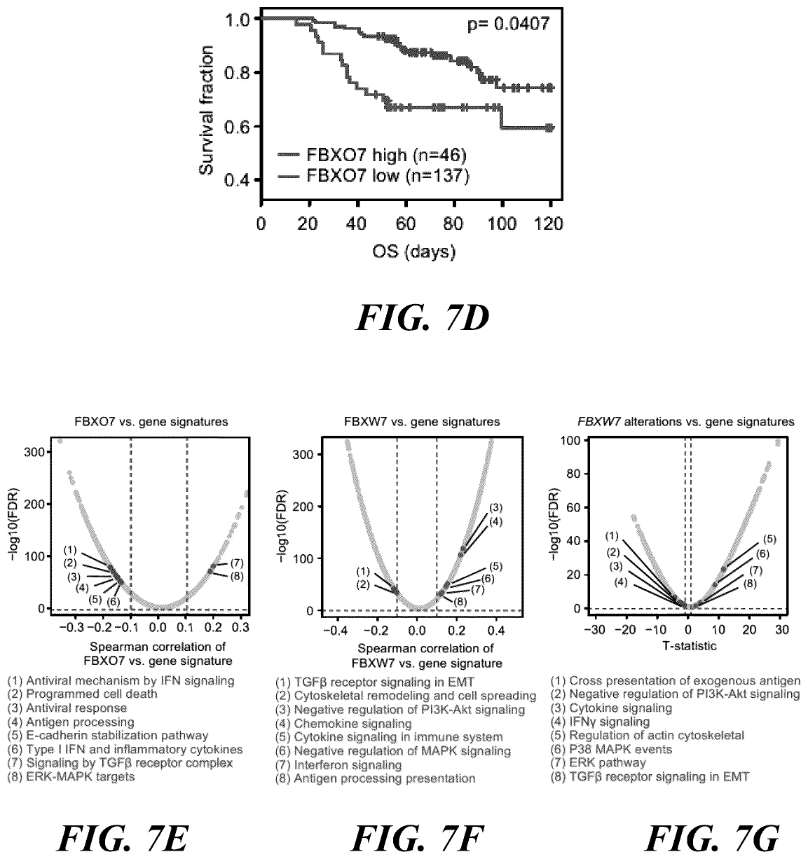

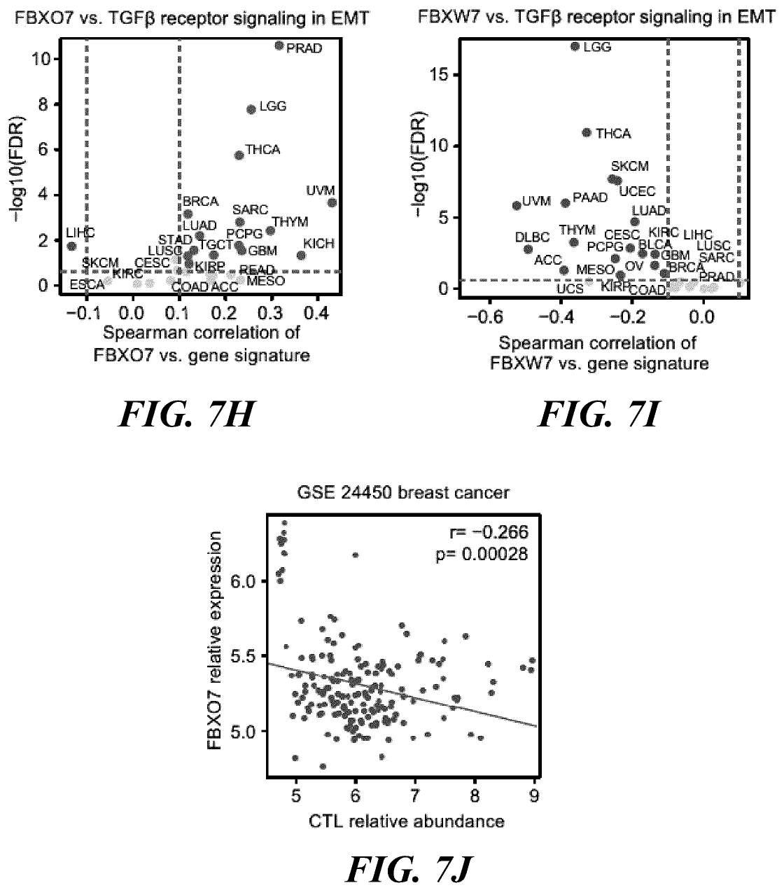

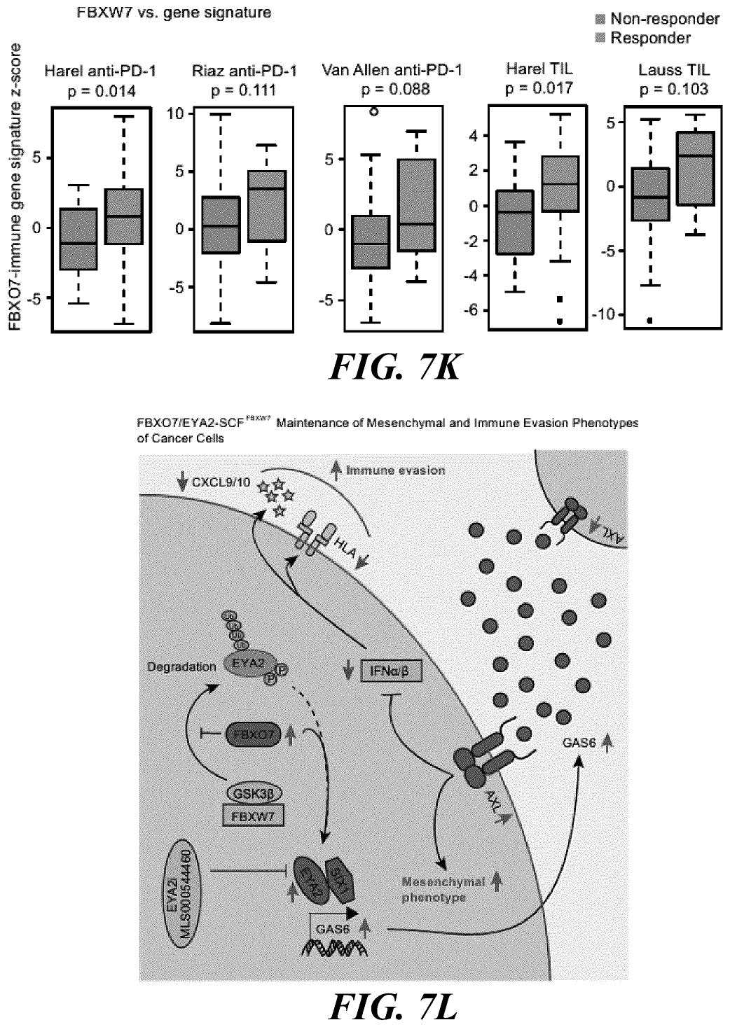

To establish clinical relevance of FBXO7, we interrogated public transcriptomic datasets to investigate if FBXO7 associated with mesenchymal phenotypes and immunotherapy responses in cancer patients. FBXO7 was overexpressed across many cancer types compared to normal adjacent tissue (FIG. 7A). In breast cancer, increased FBXO7/EYA2 expression associated with basal-like and TNBC tumors (FIGS. 7B and 7C), which exhibit enhanced EMT and CSC characteristics. High FBXO7 expression associated with reduced survival of these patients (FIG. 7D).

Interrogation of The Cancer Genome Atlas (TCGA) revealed FBXO7 expression correlated with several EMT-related pathways, including TGFβ receptor and MAPK signaling, and inversely correlated with antitumor immune response pathways, including antigen processing, programmed cell death, and type I IFN and inflammatory cytokines (FIG. 7E). Consistent with SCFFBXW7 antagonizing FBXO7 functions, FBXW7 low-expression and Fbxw7 alterations associated with increased EMT-related signaling and decreased immune-stimulatory pathways (FIGS. 7F and 7G). TGFβ receptor signaling in EMT, a pathway induced by EYA2-SIX1, correlated with FBXO7 expression and inversely with FBXW7 expression in various cancer types (FIGS. 7H and 7I), further supporting antagonistic functions.

To investigate the clinical relevance of FBXO7/EYA2 targeting for cancer immunotherapy, patient data were analyzed to determine if FBXO7 expression correlated with an immune suppressive tumor microenvironment. FBXO7 expression inversely correlated with gene expression signatures associated with infiltration of cytotoxic T lymphocytes (CTLs) in breast cancer (FIG. 7J). To evaluate if FBXO7 expression could predict cancer patients that might respond to immunotherapy, an FBXO7-immune gene signature was generated and evaluated the predictive value of this signature score across multiple public cancer immunotherapy datasets, including anti-PD-1 and tumor-infiltrating T cell (TIL) therapies. Responders to these immune therapies expressed higher levels of the FBXO7-immune gene signature compared to non-responders across the datasets (FIG. 7K). These data suggest that targeting FBXO7/EYA2 could have therapeutic potential by reducing mesenchymal and immune evasion phenotypes, thereby attenuating tumor growth and metastasis, and enhancing antitumor immunity and immunotherapy responses (FIG. 7L).

### Example 8: Screening a Compound Library to Identify Compounds that Interacts with FBXO7

The initial structure of human FBXO7 protein is obtained. Molecular Dynamic (MD) simulations. First, the protein structure files (PSF) for MD simulations is prepared by visual molecular dynamics after removing crystallographic water molecules and adding hydrogen atoms. In the first 10000 steps of minimization, the backbone is fixed. Further 10,000 steps of minimization are performed on all atoms with no pressure control. Subsequently, the system is brought to physiological temperature (310 K) by 10 K increments with 10 ps simulation for each increment in which alpha-carbons are restrained. The constraint scaling decreases from 1 to 0.25 kcal/(mol A2) in 0.25 increments, with each increment being 5,000 steps. Further 90,000 steps with zero constraint scaling are performed as the final part of energy minimization before RMSD of protein is converged and stabilized. MD simulation for equilibrium is performed using the Langevin dynamics at 310 K with a damping coefficient of 5 ps-1, 1 atm constant pressure and the Langevin piston period and decay of 100 and 50 fs respectively. The bonded interactions, the van der Waals interaction with I2A cutoff, and the long-range electrostatic interactions with the particle-mesh Ewald (PME) are considered in the calculations of the forces acting on the system. The final structure of the FBXO7 after 10 ns of simulation is used as the receptor for docking step.

### Docking Setup

After MD simulations, water molecules of the system are deleted and a binding region corresponding to FBXO7:EYA2 protein interaction domain is selected. A library of small molecules is obtained. AutoDock Vina is used to estimate protein-ligand affinity in the binding region and predicting the best binding conformations of the compounds. The protein-compound interactions of the top 500 hit compounds are visually examined. The threshold value for hydrogen bonding is set as 3.4 A and the accessible surface area is created considering a radius of 1.4 A for solvent molecules. A final number of 100 compounds with affinities ranging from −7 to −10 kcal/mol are reached by considering features such as favorable shape complementarity and diversity in binding region and chemical properties. The top 100 compounds are then tested experimentally for treatment of immune checkpoint blockade therapy resistant cancer.

To identify the lead compounds from the top 100 compounds, in vitro, ex vivo, and in vivo experiments comprising a cell-based assay for measuring cell proliferation and AXL expression, or tumor implanted animal model for tumor size measurement are conducted. In some cases, the lead compounds can be co-treated with FBXO7 inhibitors, EYA2 phosphatase inhibitors or both as described herein.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

### Example 9: EYA2 Phosphatase Assay

Eya phosphatase activity is measured in 50 μL reactions, using black, 96-well, half-volume microtiter plates (GreinerBio-one) with OMFP (3-O-methylfluoresceinphosphate, Sigma-Aldrich) as a substrate, which is converted to a fluorescent product OMF upon dephosphorylation. First, 25 μL of Eya2 ED, Eya2 ED F290Y, or FL Eya2 (final concentration 150 nM) in the reaction buffer (25 mM HEPES (pH6.5), 50 mM NaCl, 5 mM MgCl2, 0.33% BSA, and 5 mM DTT) is added into wells, then 0.5 μL of each compound, that is serially diluted in DMSO at eight different concentrations, is added to the corresponding wells. The plate is incubated for 10 minutes at room temperature. Reactions are started with the addition of OMFP (final concentration 50 μM), incubated for 1 hour at room temperature in the dark, then terminated with the addition of EDTA (final concentration 75 mM). Phosphatase assays for different Eya family members are carried out using 150 nM Eya1 ED, Eya2 ED, or Eya4 ED, 50 μM OMFP, and 100 μM of each compound. Fluorescence intensity is measured at 485/515 nm excitation/emission on a Fluoromax-3 plate reader (Horiba Jobin Yvon). The data were analyzed and IC50s were obtained using the Prism software (GraphPad).

1. A method of treating cancer in a subject comprising administering a therapeutically effective amount of treatment to the subject, wherein the treatment comprises i) a F-box only protein 7 (FBXO7) inhibitor, an Eyes absent homology 2 (EYA2) inhibitor, or both, ii) a chemical entity that inhibits the interaction between FBXO7 and EYA2, or iii) a chemical entity that interferes with the stabilization of EYA2 by FBXO7.

2. (canceled)

3. (canceled)

4. The method of claim 1, wherein the treatment comprises the FBXO7 inhibitor and the EYA2 inhibitor.

5. The method of claim 4, wherein the EYA2 inhibitor is an EYA2 Tyr phosphatase inhibitor.

6. The method of claim 5, wherein the EYA2 Tyr phosphatase inhibitor is: 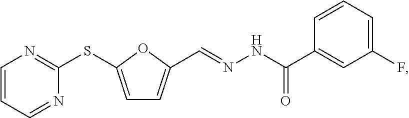 [SMILES: O=C(N/N=C/c1ccc(Sc2ncccn2)o1)c1cccc(F)c1]
or the EYA2 Tyr phosphatase inhibitor is:

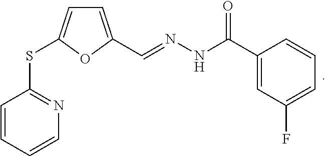 [SMILES: O=C(N/N=C/c1ccc(Sc2ccccn2)o1)c1cccc(F)c1 ; "."]

7. (canceled)

8. The method of claim 4, wherein the FBXO7 inhibitor is a knockdown mechanism comprising at least one of an RNA interference (RNAi), a small interfering RNA (siRNA), a short hairpin RNA (shRNA), a hairpin siRNA, a precursor microRNA (pre-miRNA) or a microRNA (miRNA), a zinc finger nuclease (ZFN), a bacterial RNA-guided endonuclease, and/or a TAL-effector nuclease (TALEN) directed towards FBXO7 gene.

9. The method of claim 8, wherein the knockdown mechanism comprises siRNA, or wherein the knockdown mechanism comprises shRNA.

10-82. (canceled)

83. A method for identifying an agent for treating immune checkpoint blockade therapy resistant cancer, comprising:
a. contacting F-box only protein 7 (FBXO7) with a test agent;
b. determining whether the test agent interacts with FBXO7; and
c. subjecting the test agent to an in vitro, ex vivo or in vivo test, wherein a test agent that interacts with FBXO7 reduces or prevents cell proliferation.

84. The method of claim 83, wherein determining whether the test agent interacts with FBXO7 comprising measuring a Green Fluorescent Protein (GFP) signal.

85. The method of claim 84, wherein the GFP signal shows reduction over an increasing dose of the test agent.

86. The method of claim 83, wherein determining whether the test agent interacts with FBXO7 comprises measuring growth of cells over time.

87. The method of claim 86, wherein there is a decrease of growth of cells over time relative to nontreatment.

88. The method of claim 83, wherein determining whether the test agent interacts with FBXO7 comprises measuring tumor size in one or more mice treated with the test agent.

89. The method of claim 88, wherein there is a decrease of tumor size in at least one or more mice treated with the test agent relative to nontreatment.

90. The method of claim 83, wherein determining whether the test agent interacts with FBXO7 comprises measuring levels of JUN, MYC, ZEB1, TUBA1C, CDK6, AKT3, or GAS6 protein.

91. The method of claim 90, wherein there is an abolition or decrease of protein level relative to nontreatment.

92-133. (canceled)

134. A method of screening a compound library to identify compounds which inhibit an interaction between FBXO7 and EYA2, comprising:
a. obtaining a library comprising a plurality of compounds;
b. submitting each compound of the compound library to an in vitro assay reporting a signal, wherein the signal indicates the compound inhibits an interaction between FBXO7 and EYA2;
c. measuring the signal over a range of increasing concentration of the compound;
d. obtaining an EC₅₀ or IC₅₀ output value; and
e. ordering the compounds based on the output value.

135. The method of claim 134, wherein the signal indicates a GAS6 protein level.

136. The method of claim 134, wherein the signal indicates a EYA2 phosphatase activity level.

137. The method of claim 136, wherein the EYA2 phosphatase activity level is determined using fluorescence intensity.

138. The method of claim 136, wherein the EYA2 phosphatase activity level is determined by measuring the fluorescence intensity of 3-O-methylfluorescein.


SEQUENCE LISTING (1 sequence)

SEQ ID NO: 1 - protein, 538 aa, Homo sapiens
MVELVISPSLTVNSDCLDKLKFNRADAAVWTLSDRQGITKSAPLRVSQLFSRSCPRVLPRQPSTAMAAYGQTQYSAGIQQATPYTAYPPPAQAYGIPSFSIKTEDSLNHSPGQSGFLSYGSSFSTSPTGQSPYTYQMHGTTGFYQGGNGLGNAAGFGSVHQDYPSYPGFPQSQYPQYYGSSYNPPYVPASSICPSPLSTSTYVLQEASHNVPNQSSESLAGEYNTHNGPSTPAKEGDTDRPHRASDGKLRGRSKRSSDPSPAGDNEIERVFVWDLDETIIIFHSLLTGTFASRYGKDTTTSVRIGLMMEEMIFNLADTHLFFNDLEDCDQIHVDDVSSDDNGQDLSTYNFSADGFHSSAPGANLCLGSGVHGGVDWMRKLAFRYRRVKEMYNTYKNNVGGLIGTPKRETWLQLRAELEALTDLWLTHSLKALNLINSRPNCVNVLVTTTQLIPALAKVLLYGLGSVFPIENIYSATKTGKESCFERIMQRFGRKAVYVVIGDGVEEEQGAKKHNMPFWRISCHADLEALRHALELEYL